(19) United States
(12) Patent Application Publication
(10) Pub. No.: US 2025/0090835 A1
(43) Pub. Date: Mar. 20, 2025

(54) Tri-Axis Accelerometers for Patient Physiologic Monitoring and Closed Loop Control of Implantable Ventricular Assist Devices

(71) Applicant: TC1 LLC, St. Paul, MN (US)

(72) Inventors: Daniel I. Harjes, Carlisle, MA (US); Russell Anderson, Hopkins, MN (US); Pritika Toutam, Plymouth, MN (US); Brian Kimball, Medford, MA (US)

(73) Assignee: TC1 LLC, St. Paul, MN (US)

(21) Appl. No.: 18/959,115

(22) Filed: Nov. 25, 2024

Related U.S. Application Data
(63) Continuation of application No. PCT/US2023/022590, filed on May 17, 2023.
(60) Provisional application No. 63/346,007, filed on May 26, 2022.

(51) Int. Cl. A61M 60/515; A61M 60/178; A61M 60/216; A61M 60/422

(52) U.S. Cl. CPC A61M 60/515; A61M 60/178; A61M 60/216; A61M 60/422; A61M 2205/3365; A61M 2205/50; A61M 2230/06; A61M 2230/42; A61M 2230/62; A61M 2230/63

(57) ABSTRACT

Blood circulation assist systems include a ventricular assist device (VAD), a remote accelerometer, and a controller that controls operation of the VAD based on output of the remote accelerometer. A blood circulation assist system includes a VAD, a controller, and a remote accelerometer. The VAD includes an impeller. The remote accelerometer is configured to generate a remote accelerometer output indicative of accelerations measured by the remote accelerometer. The implanted controller controls a rotation speed of the impeller based on the remote accelerometer output.

### CROSS REFERENCE TO RELATED APPLICATION DATA

The present application is a Continuation of PCT/US2023/022590 filed May 17, 2023; which claims the benefit of U.S. Provisional Appln No. 63/346,007 filed May 26, 2022, the full disclosures which are incorporated herein by reference in their entirety for all purposes.

### BACKGROUND

Ventricular assist devices, known as VADs, are used for both short-term (i.e., days, months) and long-term blood circulation assistance (i.e., years or a lifetime) where a patient's heart is incapable of providing adequate circulation, commonly referred to as heart failure or congestive heart failure. According to the American Heart Association, more than five million Americans are living with heart failure, with about 670,000 new cases diagnosed every year. People with heart failure often have shortness of breath and fatigue. Years of living with blocked arteries and/or high blood pressure can leave a heart too weak to pump enough blood to the body. As symptoms worsen, advanced heart failure develops.

A patient suffering from heart failure may use a VAD while awaiting a heart transplant or as a long term destination therapy. A patient may also use a VAD while recovering from heart surgery. Thus, a VAD can supplement a weak heart (i.e., partial support) or can effectively replace the natural heart's function.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Blood circulation assist systems and related methods employ a remote accelerometer that measures accelerations within a patient's body away from an implanted ventricular assist device. In some embodiments, an implanted transcutaneous energy system (TETS) power receiver unit includes the remote accelerometer and the remote accelerometer measures induced accelerations of the TETS power receiver unit. In some embodiments, an implanted controller unit includes the remote accelerometer and the remote accelerometer measures induced accelerations of the implanted unit. In many embodiments, the measured accelerations are processed for use in controlling operation of the VAD, generating patient monitoring data, and/or generating VAD monitoring data. In some embodiments, the measured accelerations are processed to measure patient activity level, which is used to control the output level of the VAD based on the patient activity level. In such embodiments, the output level of the VAD can be increased in response to an increase in the patient activity level and decreased in response to a decrease in the patient activity level. In some embodiments, the measured accelerations are processed to track the patient's cardiac cycle timing, which is used to control variation in output of the VAD in synchronization with the patient's cardiac cycle timing. In some embodiments, the measured accelerations are used to generate patient monitoring data and/or VAD monitoring data. By controlling operation of the VAD based on patient activity level and/or in synch with the patient's cardiac cycle timing, the circulatory support provided is better tailored to the needs of the patient. The availability of the patient monitoring data and/or the VAD tracking data may enable increased ability to diagnose patient health issues and/or VAD operational problems. In some embodiments in which the VAD includes power transistors used to control flow of currents to a motor stator of the VAD, the remote accelerometer is located away from the power transistors and electrical noise generated by power transistors. As a result of being located away from the electrical noise, the remote accelerometer may generate an output signal that more accurately reflects the induced accelerations of the remote accelerometer.

Thus, in one aspect, a blood circulation assist system includes a ventricular assist device (VAD), a transcutaneous energy transfer system (TETS) power receiver, a controller, and a remote accelerometer. The VAD includes an inlet, an outlet, an impeller, and a motor stator operable to rotate the impeller to pump a blood flow. The inlet is configured for coupling with a ventricle of a patient to receive the blood flow from the ventricle. The outlet is configured for coupling with a blood vessel of the patient to transfer the blood flow to the blood vessel. The TETS power receiver is configured to be implanted and receive energy transmitted by an external TETS transmitter. The controller is configured to be implanted, process a remote accelerometer output, and control a rotation speed of the impeller based on the remote accelerometer output. The remote accelerometer is configured to generate the remote accelerometer output. In some embodiments, the TETS power receiver includes the remote accelerometer and the remote accelerometer output is indicative of the accelerations of the TETS power receiver. In other embodiments, the controller includes the remote accelerometer and the remote accelerometer output is indicative of the accelerations of the controller. In many embodiments, the remote accelerometer output is indicative of accelerations in three orthogonal directions.

The remote accelerometer can be integrated into the TETS power receiver in any suitable manner. For example, the remote accelerometer can be mounted to an inner surface of a TETS power receiver housing that forms an outer surface of the TETS power receiver. The TETS power receiver can include a TETS power receiver coil that is enclosed within the TETS power receiver housing. The TETS power receiver housing can be made from any suitable material or combination of materials. For example, the TETS power receiver housing can include a titanium panel that includes the inner surface of the TETS power receiver housing and the outer surface of the TETS power receiver. In some embodiments, the TETS power receiver includes a TETS power receiver printed circuit board assembly (PCBA) and the remote accelerometer is mounted to the TETS power receiver PCBA.

In many embodiments, the TETS power receiver and the controller are configured to be implanted in suitable locations that are spaced apart from the VAD. In many embodiments, the blood circulation assist system includes a TETS power receiver connection cable that connects the TETS power receiver to the controller. In some embodiments, the TETS power receiver is configured for implantation in a pectoral region of the patient. In many embodiments, the blood circulation assist system includes a controller connection cable that connects the controller to the VAD. In some embodiments, the controller is configured for implantation in an abdominal wall region of the patient.

In many embodiments, the controller is configured to process the remote accelerometer output to determine a range of parameters. For example, the controller can be configured to process the remote accelerometer output to determine a heart rate of the patient and control the rotation speed of the impeller based on the heart rate. The controller can be configured to process the remote accelerometer output to determine a ventricular contraction magnitude of the patient. The controller can be configured to process the remote accelerometer output to determine a cardiac output magnitude of the patient. The controller can be configured to process the remote accelerometer output to determine a valve opening timing of the patient. The controller can be configured to process the remote accelerometer output to monitor for a valve disorder of the patient. The controller can be configured to process the remote accelerometer output to determine a respiration rate of the patient. The controller can be configured to process the remote accelerometer output to monitor for an occurrence of pump thrombosis in the VAD. The controller can be configured to process the remote accelerometer output to monitor for an occurrence of an occlusion in the VAD. The controller can be configured to process the remote accelerometer output to monitor for an occurrence of an instability of the impeller. The controller can be configured to process the remote accelerometer output to monitor an orientation of the patient. The controller can be configured to process the remote accelerometer output to determine when the patient is prone. The controller can be configured to process the remote accelerometer output to determine when the patient is supine. The controller can be configured to process the remote accelerometer output to determine an angle of recline when the patient is supine. The controller can be configured to process the remote accelerometer output to determine when the patient is sitting. The controller can be configured to process the remote accelerometer output to determine when the patient is standing. The controller can be configured to process the remote accelerometer output to monitor for a fall of the patient. The controller can be configured to process the remote accelerometer output to monitor for a syncope of the patient. The controller can be configured to process the remote accelerometer output to determine whether the patient is active or at rest. The controller can be configured to process the remote accelerometer output to determine a wellness indicator for the patient.

In some embodiments, the controller is configured to process the remote

accelerometer output to detect a cardiac cycle timing of the patient. The cardiac cycle timing can include a heart rate and/or a time of occurrence for each of one or more cardiac cycle events. The controller can be configured to vary the rotation speed of the impeller in sync with the cardiac cycle timing. For example, the controller can be configured to increase the rotation speed of the impeller to during ventricular systole. The controller can be configured to process the remote accelerometer output to detect a time of occurrence of at least one heart sound and detect timing of ventricular systole based on the time of occurrence of the at least one heart sound. The at least one heart sound can include a sound of closure of at least one atrioventricular valve of the patient and/or a sound of closure of at least one semilunar valve of the patient. The controller can be configured to vary the rotation speed of the impeller over a target cardiac cycle based on detected timing of one or more cardiac cycles that occur prior to the target cardiac cycle. The controller can be configured to vary the rotation speed of the impeller over a target cardiac cycle based on detected timing of the target cardiac cycle.

In some embodiments, the controller is configured to process the remote accelerometer output to measure an activity level of the patient and control the rotation speed of the impeller based on the activity level. For example, the controller can be configured to process the remote accelerometer output to measure a respiration rate for the patient and/or a diaphragm contraction for the patient and base the activity level on the respiration rate and/or the diaphragm contraction. The controller can be configured to process the remote accelerometer output to measure an activity level of the patient and control the rotation speed of the impeller based on the activity level. The controller can be configured to process the remote accelerometer output to measure a respiration rate and/or a diaphragm contraction and base the activity level on the respiration rate and/or the diaphragm contraction.

In another aspect, a blood circulation assist system includes a VAD, a TETS power receiver, a controller, and a remote accelerometer. The VAD includes a housing that defines a blood flow channel, an inlet, an outlet, an impeller disposed within the blood flow channel, a motor stator, and a VAD accelerometer. The motor stator is operable to rotate the impeller to pump a blood flow in a patient. The inlet is configured for coupling with a ventricle of a heart to receive the blood flow from the ventricle. The outlet is configured for coupling with a blood vessel to transfer the blood flow to the blood vessel. The VAD accelerometer is configured to generate a VAD accelerometer output indicative of accelerations of the VAD. The TETS power receiver is configured to be implanted and receive energy transmitted by an external TETS transmitter. The controller is configured to be implanted, process the VAD accelerometer output and a remote accelerometer output, and control a rotation speed of the impeller based on at least one of the VAD accelerometer output and the remote accelerometer output. The remote accelerometer is configured to generate the remote accelerometer output. In some embodiments, the TETS power receiver includes the remote accelerometer and the remote accelerometer output is indicative of accelerations of the TETS power receiver. In other embodiments, the controller includes the remote accelerometer and the remote accelerometer output is indicative of accelerations of the controller.

In many embodiments, the VAD is configured to be mounted to a heart wall of the heart. The controller can be configured to process the VAD accelerometer output to monitor motion of the heart wall to detect a cardiac cycle timing of the heart and control the rotation speed of the impeller based on the cardiac cycle timing.

In some embodiments, the TETS power receiver includes the remote accelerometer and is configured for implantation in a pectoral region of the patient. The controller can be configured to process the remote accelerometer output to determine a respiration rate of the patient and control the rotation speed of the impeller further based on the respiration rate.

In some embodiments, the VAD includes control electronics configured to control drive currents supplied to the motor stator to rotate the impeller. In some embodiments, the drive currents supplied to the motor stator are further used to magnetically levitate the impeller.

In some embodiments, the controller is configured to determine a posture of the patient. For example, the controller can be configured to determine the posture of the patient based on the remote accelerometer output and/or the VAD accelerometer output.

In another aspect, a blood circulation assist system includes a ventricular assist device (VAD), a controller, and a remote accelerometer. The VAD includes an inlet, an outlet, an impeller, and a motor stator operable to rotate the impeller to pump a blood flow. The inlet is configured for coupling with a ventricle of a patient to receive the blood flow from the ventricle. The outlet is configured for coupling with a blood vessel of the patient to transfer the blood flow to the blood vessel. The controller is configured to process a remote accelerometer output indicative of accelerations of the patient at a remote accelerometer location and control a rotation speed of the impeller based on the remote accelerometer output. The remote accelerometer is configured to generate the remote accelerometer output. The remote accelerometer is configured to be implanted in the patient at the remote accelerometer location. The remote accelerometer location is separated from the VAD to isolate the remote accelerometer from noise generated by the VAD. The remote accelerometer location can be any suitable location that is separated from the VAD to isolate the remote accelerometer from noise generated by the VAD. For example, in some embodiments, the remote accelerometer location is within a pectoral region of the patient. In some embodiments, the remote accelerometer location is within an abdominal wall region of the patient.

For a fuller understanding of the nature and advantages of the present invention, reference should be made to the ensuing detailed description and accompanying drawings.

### BRIEF DESCRIPTION OF THE DRAWINGS

FIG. 1 illustrates an implantable blood circulation assist system that includes a ventricular assist device (VAD), an implantable controller, and a transcutaneous energy transfer system (TETS) power receiver, in accordance with embodiments.

FIG. 2 illustrates the blood circulation assist system of FIG. 1 implanted within a patient.

FIG. 3 illustrates some options for integration of a remote accelerometer into the TETS power receiver of the blood circulation assist system of FIG. 1.

FIG. 4 is a schematic diagram of an embodiment of the TETS power receiver of the blood circulation assist system of FIG. 1.

FIG. 5 illustrates some options for integration of a remote accelerometer into the implantable controller of the blood circulation assist system of FIG. 1.

FIG. 6 is a schematic diagram of an embodiment of the implanted system controller of the blood circulation assist system of FIG. 1.

FIG. 7 is a simplified schematic diagram of a method of operating the blood circulation assist system of FIG. 1 utilizing accelerations measured via the remote accelerometer, in accordance with many embodiments.

FIG. 8 illustrates synchronization of speed variation of the VAD of the blood circulation assist system of FIG. 1 with ventricular systole based on heart sounds, ventricular wall motion, and/or pump operating parameters, in accordance with many embodiments.

FIG. 9 is an exploded view of implanted components of the blood circulation assist system of FIG. 1.

FIG. 10 is an illustration of the VAD of the blood circulation assist system of FIG. 1 attached to the patient's heart to augment blood pumping by the patient's left ventricle.

FIG. 11 is a cross-sectional view of the VAD of the blood circulation assist system of FIG. 1.

FIG. 12 is an illustration of an embodiment of a control unit for the VAD of the blood circulation assist system of FIG. 1.

FIG. 13 is a heart-side view of the control unit of FIG. 12 showing a VAD accelerometer included in the control unit, in accordance with many embodiments.

FIG. 14 is a schematic diagram of an embodiment of the VAD of the blood circulation assist system of FIG. 1.

FIG. 15 is a plot of example raw accelerations of the VAD generated via the VAD accelerometer of the blood circulation assist system of FIG. 1.

FIG. 16 is a plot of mean normalized accelerations of the VAD generated from the raw accelerations of FIG. 15.

FIG. 17 is a plot of velocities of the VAD generated from the accelerations of FIG. 15.

FIG. 18 is a plot of displacements of the VAD generated from the velocities of FIG. 17.

FIG. 19 is a schematic diagram of external components of the blood circulation assist system of FIG. 1.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

Many existing ventricular assist devices essentially operate in a fixed speed mode. In such ventricular assist devices, an impeller of the ventricular assist device (VAD) is rotated at a constant rotational speed that is set by a clinician. Many existing VADs employ a centrifugal or axial flow blood pump with a head/flow curve (HQ curve) that shows an inverse relationship between blood flow rate through the VAD and pressure change across the VAD at every rotational speed of the impeller. The inverse relationship between blood flow rate through the VAD and pressure change across the VAD provides some level of physiologic flow response since the blood flow rate through the VAD increases in response to a decrease in the pressure change across the VAD. The level of physiologic flow response provided by a VAD operated at constant speed, however, is small relative to the native heart's ability to adjust cardiac output between rest and exercise states (Native˜4-25 L/min, VAD˜3-6 L/min).

In many embodiments, a blood circulation assist system includes a VAD and a remote accelerometer that is implanted to be spaced apart from the VAD. The remote accelerometer measures induced accelerations of the remote accelerometer that are processed to by the blood circulation assist system to monitor physiological demand of the patient. The blood circulation assist system automatically adjust VAD impeller rotational speed in response to the physiologic demand of the patient. In many embodiments, the blood circulation assist system automatically reduces the VAD impeller rotational speed during sleep/rest to prevent ventricular suction events. Automatically reduces the VAD impeller rotational speed during sleep/rest accommodates the use of a higher mean VAD impeller rotational speed to provide enhanced perfusion/support when the patient is not asleep or at rest. In many embodiments, the blood circulation assist system automatically increases the VAD impeller rotational speed during a more active patient state (e.g., during exercise) to provide higher than mean perfusion/support.

In many embodiments, the blood circulation assist system periodically reduces VAD impeller rotational speed to produce periodic opening of a ventricular valve (i.e., aortic valve, pulmonary valve) with the goal of inhibiting development of ventricular valve insufficiency. For example, aortic insufficiency may be caused by structure failure of the aortic valve resulting from prolonged closure of the aortic valve produced via the use of a fixed speed left ventricular assist device (LVAD). It has been suggested that prolonged closure of the aortic valve causes biomechanical deterioration of the aortic valve.

In many embodiments, a remote accelerometer is employed in a blood circulation assist system. The remote accelerometer is configured to generate an output signal that is indicative of induced accelerations of the remote accelerometer. The blood circulation assist system processes the output signal to monitor physiologic state of the patient, which can be used for enhanced patient diagnostics and/or closed loop control of VAD impeller rotational speed based on the physiologic state of the patient. In some embodiments, the VAD impeller rotational speed is increased or decreased as a function of physical activity of the patient within a preset range of values. The modulation of the VAD impeller rotational speed can be customized to the patient based on any suitable patient parameter or combination of patient parameters such as, for example, patient size, range of motion, and/or physical activity. In some embodiments, the blood circulation assist system includes an implanted transcutaneous energy transfer system (TETS) power receiver that includes the remote accelerometer. The TETS power receiver can be implanted in any suitable location within the patient such as, for example, in a pectoral region of the patient. In some embodiments, the blood circulation assist system includes an implanted controller that includes the remote accelerometer. The controller can be implanted in any suitable location within the patient such as, for example, in an abdominal region of the patient.

The blood circulation assist system can be configured to process the accelerations measured by the remote accelerometer to determine any suitable number of a range of possible physiologic states of the patient and/or conditions of the VAD. For example, the physiologic states of the patient that can be determined include patient position data, patient activity level, patient events, one or more wellness indicators, and cardiac cycle timing. The conditions of the VAD that can be determined include detection of occurrences of pump thrombus and/or occlusion of the VAD inlet, VAD outlet, and/or VAD impeller. The patient position data can be indicative of whether the patient is prone, supine, sitting, or standing. If the patient is supine, an angle of recline of the patient can be measured. The angle of recline of the patient when supine may be indicative of an extent of heart failure since the angle of recline may be indicative of the number of pillows required to allow circulation while sleeping. The patient activity level can be indicative of whether the patient is at rest or is active. If the patient is active, the patient's activity level can be quantified using any suitable approach. The patient events that can be detected can include, for example, a fall of the patient and a syncope of the patient. One example wellness indicator that can be determined is a distance traveled during a timed walk (e.g., a six minute hall walk). The patient's cardiac cycle timing can be monitored via processing of the accelerations measured by the remote accelerometer to detect heart sounds (e.g., S1, S2, S3, S4). The detected heart sounds can be used to determine heart rate, ventricular contractility, cardiac output, and valve opening timing. The accelerations measured by the remote accelerometer can be processed to detect valve disorders (e.g., stenosis, regurgitation, etc.) and respiration rate.

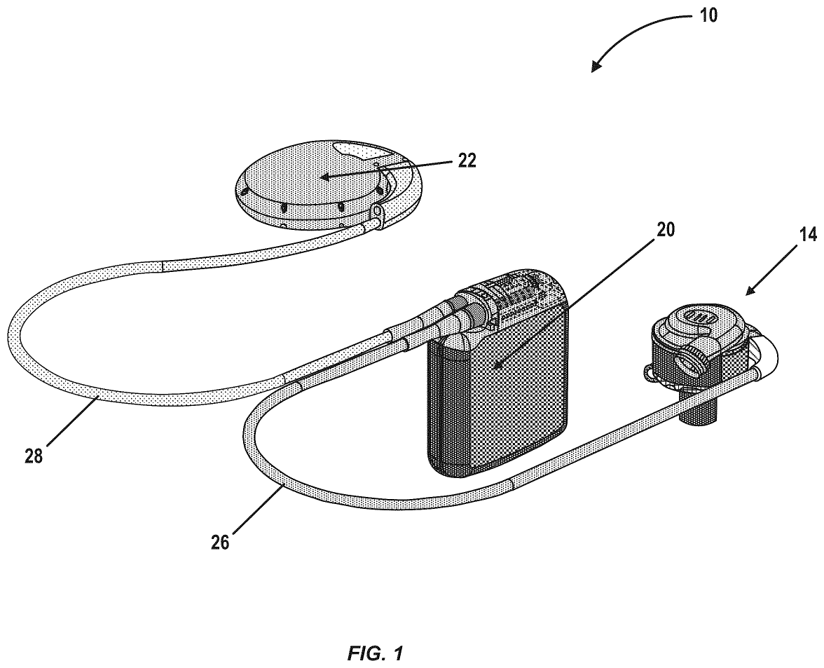

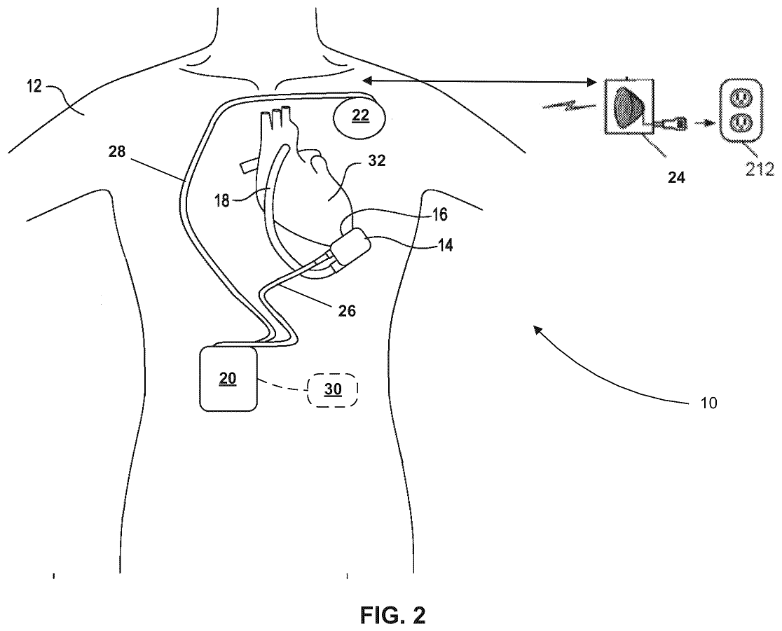

Referring now to the drawings, in which like reference numerals represent like parts throughout the several views, FIG. 1 illustrates implantable components of a blood circulation assist system 10, in accordance with embodiments. FIG. 2 illustrates the blood circulation assist system 10 implanted in a patient 12. The system 10 includes a ventricular assist device (VAD) 14, a ventricular cuff 16, an outflow cannula 18, an implanted controller 20, a transcutaneous energy transfer system (TETS) power receiver 22, a TETS power transmitter 24, a controller-to-VAD connection cable 26, a TETS power receiver-to-controller connection cable 28, and a remote accelerometer 30. In the embodiment illustrated in FIG. 2, the remote accelerometer 30 is implanted within the patient at a remote accelerometer location that is separated from the VAD 14 so as to isolate the remote accelerometer 30 from noise generated by the VAD, such as electrical switching noise generated by the VAD 14 by power transistors included within the VAD 14 that are used to control supply of currents to windings of a stator of the VAD 14. The remote accelerometer 30 can be configured to be implanted in the patient at any suitable location so as to isolate the remote accelerometer 30 from noise generated by the VAD 14 including, but not limited to, in the abdomen of the patient below the diaphragm of the patient or in the pectoral region. The remote accelerometer 30 can be a suitable 3-axis accelerometer (e.g., with 10 bit or 12 bit resolution) that is located in the pectoral region and generates output that is processed to discern heart sounds. The remote accelerometer 30 can be included in the controller 20 or the TETS power receiver 22 as described herein.

The VAD 14 can be attached to an apex of the left ventricle, as illustrated, or the right ventricle, or a separate VAD can be attached to each of the ventricles of the heart 32. The VAD 14 can be capable of pumping the entire flow of blood delivered to the left ventricle from the pulmonary circulation (i.e., up to 10 liters per minute). Related blood pumps applicable to the present invention are described in greater detail below and in U.S. Pat. Nos. 5,695,471, 6,071,093, 6,116,862, 6,186,665, 6,234,772, 6,264,635, 6,688,861, 7,699,586, 7,976,271, 7,997,854, 8,007,254, 8,152,493, 8,419,609, 8,652,024, 8,668,473, 8,852,072, 8,864,643, 8,882,744, 9,068,572, 9,091,271, 9,265,870, and 9,382,908, all of which are incorporated herein by reference for all purposes in their entirety. The VAD 14 can be attached to the heart 32 via the ventricular cuff 16, which can be sewn to the heart 32 and coupled to the VAD 14. In the illustrated embodiment, the output of the VAD 14 connects to the ascending aorta via the outflow cannula 18 so that the VAD 14 effectively diverts blood from the left ventricle and propels it to the aorta for circulation through the rest of the patient's vascular system.

The controller-to-VAD connection cable 26 connects the VAD 14 to the implanted controller 20, which monitors system 10 operation. Related controller systems applicable to the present invention are described in greater detail below and in U.S. Pat. Nos. 5,888,242, 6,991,595, 8,323,174, 8,449,444, 8,506,471, 8,597,350, and 8,657,733, EP 1812094, and U.S. Patent Publication Nos. 2005/0071001 and 2013/0314047, all of which are incorporated herein by reference for all purposes in their entirety.

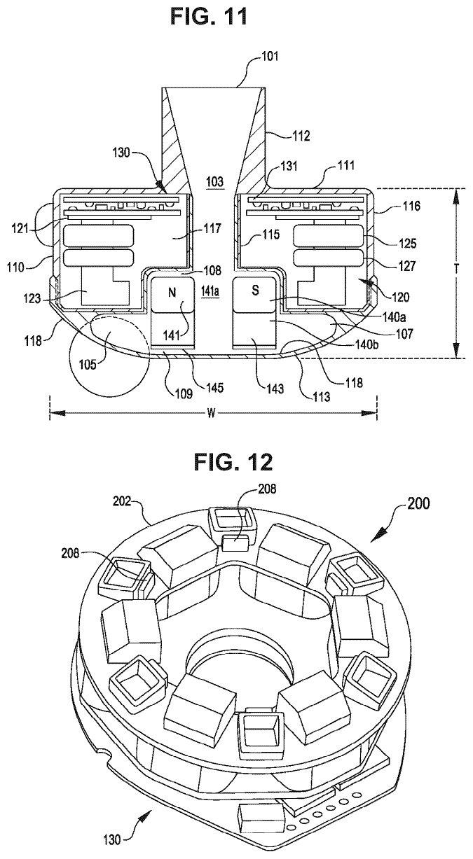

The VAD 12 includes an inlet cannula 112, a rotor/impeller 140, a motor stator 120, and an outlet opening 105 (shown in FIG. 10 and FIG. 11). When implanted, the inlet cannula 112 extends into the ventricle and the outlet opening 105 is placed in fluid communication with the blood vessel through the outflow cannula 18. The rotor/impeller 140 is controllably rotated via controlled drive currents supplied to the motor stator 120. The rate of flow of the blood flow through the VAD 14 can be controlled via controlling of the rate of rotation of the rotor/impeller 140. In many embodiments, the VAD 14 includes electronics 130 (shown in FIG. 10 through FIG. 13) that include power transistors that are controlled to control the drive currents supplied to the motor stator to control the rate of rotation of the rotor/impeller 140. In many embodiments, the power transistors of the electronics 130 also control levitation currents supplied to the motor stator to control magnetic levitation of the rotor/impeller 140.

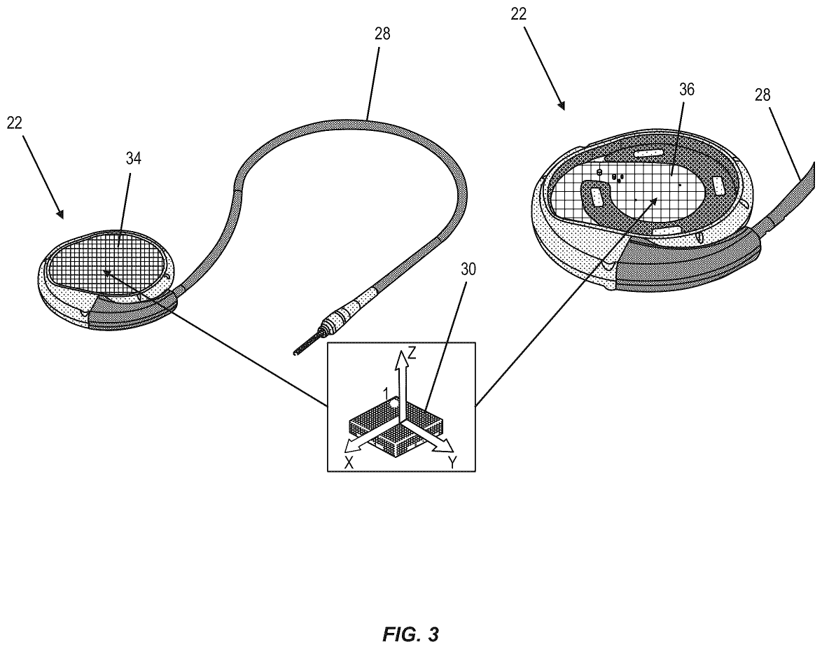

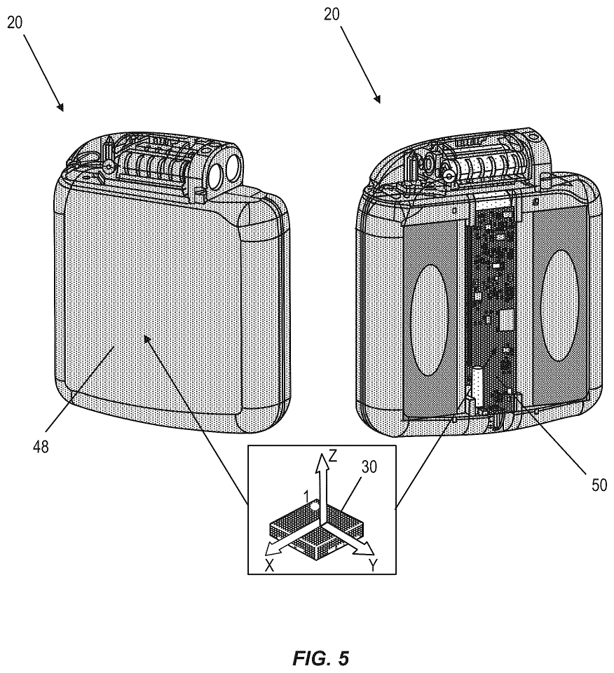

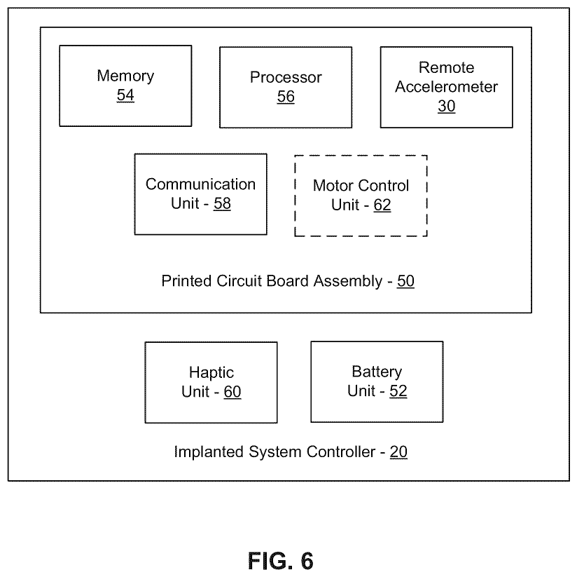

The implanted controller 20 is configured to supply power to and control operation of the VAD 14. The implanted controller 20 is configured to be implanted within the patient 12 in a suitable location spaced apart from the VAD 14. The implanted controller 20 is operatively coupled with the VAD 14 via the controller-to-VAD connection cable 26. The implantable controller 20 is configured to receive and process output from the remote accelerometer 30 (examples shown in FIG. 2, FIG. 3 and FIG. 5) for use in controlling the rate of rotation of the rotor/impeller 140 as described herein. In the embodiment shown in FIG. 5 and FIG. 6, the implantable controller 20 includes the remote accelerometer 30.

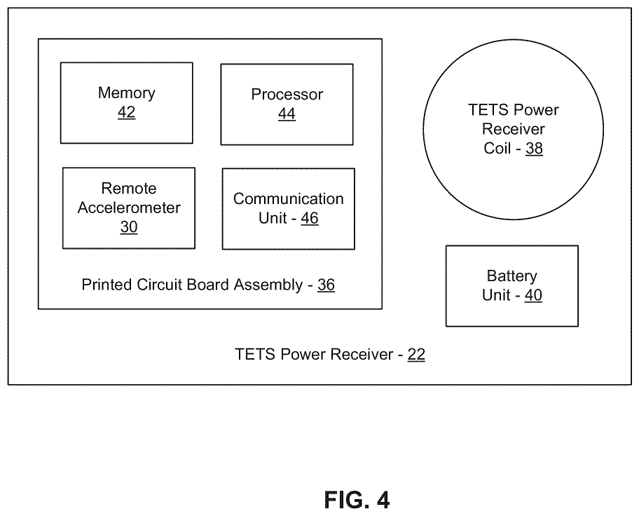

The TETS power receiver 22 is configured to receive power transmitted by the TETS power transmitter 24 for powering operation of the system 10. The TETS power receiver 22 is configured to be implanted within the patient 12 in a suitable location spaced apart from the VAD 14 and the controller 20. The TETS power receiver 22 is operatively coupled with and supplies power to the controller 20 via the TETS power receiver-to-controller connection cable 28. In the embodiment shown in FIG. 3 and FIG. 4, the TETS power receiver 22 includes the remote accelerometer 30.

FIG. 3 illustrates some options for integration of the remote accelerometer 30 into the TETS power receiver 22. In the illustrated embodiment, the TETS power receiver 22 includes a lid/cap 34 and a printed circuit board assembly (PCBA) 36. The lid/cap 34 can be made from any suitable material (e.g., titanium). In some embodiments, the remote accelerometer 30 is mounted to an inner surface of the lid/cap 34. In some other embodiments, the remote accelerometer 30 is included in the PCBA 36. Mounting the remote accelerometer 30 to the inner surface of the lid/cap 34 may provide optimal tissue coupling. In embodiments in which the remote accelerometer 30 is mounted to the inner surface of the lid/cap 34, the remote accelerometer 30 can be operatively connected to the PCBA 36 through suitable electrical interconnects for power and communications. In embodiments in which the TETS power receiver 22 includes the remote accelerometer 30, the TETS power receiver 22 can include suitable shielding to inhibit noise in the output of the remote accelerometer 30 induced by the TETS electromagnetic field generated via the power transmission from the TETS power transmitter 24 to the TETS power receiver 22.

FIG. 4 is a schematic diagram of the embodiment of the TETS power receiver 22 of FIG. 3. The TETS power receiver 22 includes the PCBA 36, a TETS power receiver coil 38, and a TETS power receiver battery unit 40. In the illustrated embodiment, the PCBA 36 includes the remote accelerometer 30, a memory 42, a processor 44, and a communication unit 46. The remote accelerometer 30, however, can alternately be attached to the lid/cap 34 and operatively coupled with the PCBA 36 via suitable electrical interconnects for power and communication as described herein. The memory 42 can store suitable instructions executable by the processor 44 for controlling electrical characteristics of the TETS power receiver coil 38 to enhance efficiency of transfer of power from the TETS power transmitter 24 to the TETS power receiver coil 38. The memory 42 can store suitable instructions executable by the processor 44 for receiving output from the remote accelerometer 30 and controlling operation of the communication unit 46 to transmit the output from the remote accelerometer 30 to the controller 20 or acceleration data generated by the processor 44 via processing of the output from the remote accelerometer 30. The TETS power receiver battery unit 40 can store energy used to operate the TETS power receiver 22, the implanted controller 20, and/or the VAD 14 during time periods when power is not being received by the TETS power receiver coil 38.

FIG. 5 illustrates some options for integration of the remote accelerometer 30 into the implantable controller 20. In the illustrated embodiment, the implanted controller 20 includes an enclosure 48 and a printed circuit board assembly (PCBA) 50. The enclosure 48 can be made from any suitable material (e.g., titanium). In one embodiment, the remote accelerometer 30 is mounted to an inner surface of the enclosure 48. In another embodiment, the remote accelerometer 30 is included in the PCBA 50. Mounting the remote accelerometer 30 to the inner surface of the enclosure 48 may provide optimal tissue coupling. In embodiments in which the remote accelerometer 30 is mounted to the inner surface of the enclosure 48, the remote accelerometer 30 can be operatively connected to the PCBA 50 through suitable electrical interconnects for power and communications.

FIG. 6 is a schematic diagram of the embodiment of the implanted controller 20 of FIG. 5. The controller 20 includes the PCBA 50, a controller battery unit 52, and a haptic unit 60. In the illustrated embodiment, the PCBA 50 includes the remote accelerometer 30, a memory 54, a processor 56, and a communication unit 58. In some embodiments, the PCBA 50 includes a motor control unit 62 configured to control drive currents supplied to the motor stator 120 of the VAD 14 to control the rotational rate of the rotor/impeller 140 to control the flow rate of the blood flow through the VAD 14. The remote accelerometer 30 can alternately be attached to the inner surface of the enclosure 48 and operatively coupled with the PCBA 36 via suitable electrical interconnects for power and communication as described herein. The memory 54 can store suitable instructions executable by the processor 56 for processing the output of the remote accelerometer 30 to monitor physiological demand of the patient, automatically adjust VAD impeller rotational speed in response to the physiologic demand of the patient, determine any suitable number of a range of physiologic states of the patient that are discernable from the output of the remote accelerometer 30, and/or monitor conditions of the VAD 14 as described herein. The controller battery unit 52 can store energy used to operate the VAD 14, the controller 20, and/or the TETS power receiver 22 during time periods when power is not being received by the TETS power receiver coil 38. The communication unit 58 can be configured to communicate control commands to the VAD 14 over the controller-to-VAD connection cable 26. The communication unit 58 can also include a suitable wireless communication unit for receiving programming updates and/or for transmitting alarms, VAD operational data, and/or patient physiologic data to an external system monitor.

The controller 20 can be configured so that the haptic unit 60 is operated to generate a haptic alarm to alert the patient that power stored in the controller battery unit 52 and/or the TETS receiver battery unit 40 has dropped below a suitable minimum threshold so that the patient can take action to use the TETS power transmitter 24 to transmit power to the TETS power receiver 22 to recharge the controller battery unit 52 and/or the TETS power receiver battery unit 40. To guard against a prolonged latent failure of the haptic unit 60, the controller 20 can periodically command operation of the haptic unit 60 and process the output of the remote accelerometer 30 to determine whether the haptic unit 60 operated properly or is in a failed state. If the controller 20 determines that the haptic unit 60 is in a failed state, the controller 20 can communicate a suitable alarm indicating the failure of the haptic unit 60 via wireless communication by the communication unit 58.

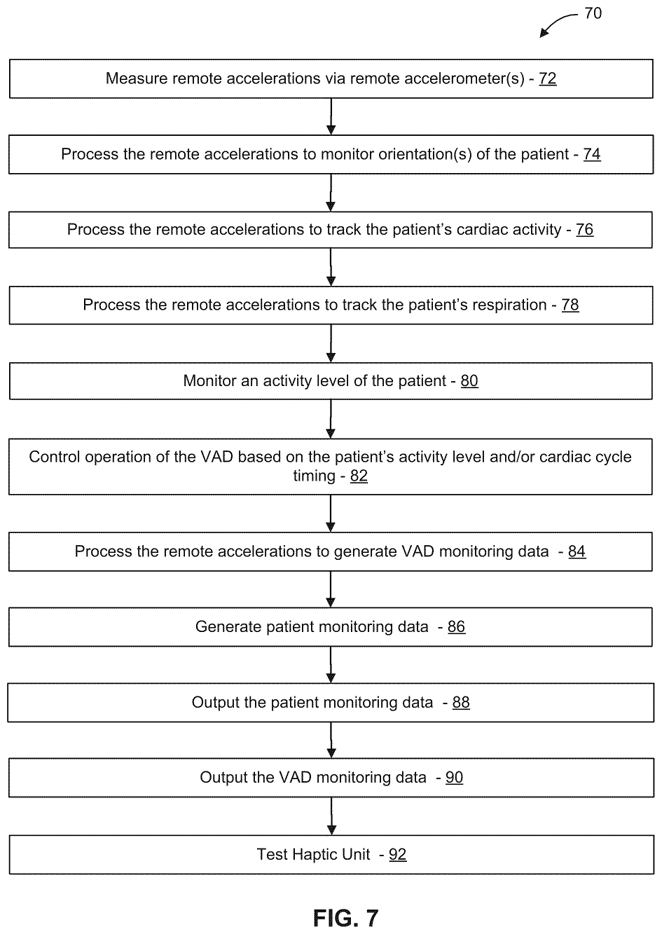

FIG. 7 is a simplified schematic diagram of a method 70 of operating a blood circulation assist system utilizing accelerations measured by a remote accelerometer. While the method 70 is described herein with respect to the blood circulation assist system 10, the method 70 can be accomplished by any suitable blood circulation assist system. The acts of the method 70 can be practiced in any suitable combination, sub-combination, and/or order.

In act 72, the remote accelerometer 30 generates output indicative of accelerations of the remote accelerometer 30. The accelerations of the remote accelerometer 30 can be induced via gravity and patient physiological activities (e.g., cardiac activity, respiration).

In act 74, the output of the remote accelerometer 30 is processed by the controller 20 to monitor orientation of the patient. In many embodiments, the output from the remote accelerometer 30 includes acceleration due to gravity and is therefore indicative of the orientation of the remote accelerometer 30, therefore also the portion of the patient 12 where the remote accelerometer 30 is located, relative to vertical. The controller can use any suitable approach 20 to process the output from the remote accelerometer 30 to determine the orientation of the remote accelerometer 30 relative to vertical. In many embodiments, the remote accelerometer 30 is configured to measure accelerations in three orthogonal directions (e.g., X-axis accelerations, Y-axis accelerations, and Z-axis accelerations). Each of the X-axis accelerations, Y-axis accelerations, and Z-axis accelerations can be processed by the controller 20 to calculate a respective running average having a suitable time period (e.g., 10 to 15 seconds) corresponding to an X-axis gravity induced acceleration, a Y-axis gravity induced acceleration, and a Z-axis gravity induced acceleration, respectively. The X-axis gravity induced acceleration, Y-axis gravity induced acceleration, and the Z-axis gravity induced acceleration define a gravity vector that indicates the orientation of the remote accelerometer 30 relative to vertical. The controller can use any suitable approach 20 to process the gravity vector to generate an indication of the orientation of the patient relative to a suitable reference axis or reference direction. For example, the gravity vector can be transformed from the axis system of the remote accelerometer 30 to a suitable patient reference axis system, for example, with a patient X-axis extending forward relative to the patient's thorax, a patient Y-axis extending to the left relative to the patient's thorax, and a patient Z-axis extending toward the top of the patient's thorax. As another example, reference gravity orientation vectors corresponding to known orientations of the patient 12 and/or the remote accelerometer 30 can be generated by placing the patient 12 and/or the remote accelerometer 30 in known orientations relative to vertical (e.g., standing upright, laying horizontal on the patient's left side, laying horizontal on the patient's right side, laying horizontal on the patient's back, and laying horizontal on the patient's stomach). The gravity vector can be compared to each of one or more of the reference gravity orientation vectors using a known approach to determine a relative angle between the gravity vector and the respective reference gravity orientation vector. The resulting relative angle(s) are indicative of the orientation of the patient 12 and/or the remote accelerometer 30 relative to the reference orientations of the patient 12 and/or the remote accelerometer 30.

In act 76, the output of the remote accelerometer 30 is processed by the controller 20 to track the patient's cardiac activity. In some embodiments, the output of the remote accelerometer 30 is processed by the controller 20 to detect and/or measure heart sounds. The heart sounds that can be detected and/or measured via the remote accelerometer 30 include a first sound (S1) generated by closing of the atrioventricular valves during ventricular contraction and a second sound (S2) generated by closing of the semilunar valves during ventricular diastole. In some patients, the occurrence of aortic insufficiency (aka aortic regurgitation) generates a corresponding sound that can be detected via the remove accelerometer 32. In some embodiments, the output of the remote accelerometer 30 is processed by the controller 20 using a suitable band-pass filter (e.g., 100 to 400 Hz) to isolate accelerations due to the heart sounds. The accelerations due to the heart sounds can then be processed by the controller 20 to detect/measure the heart sounds. The heart sounds can be used to monitor the cardiac cycle timing of the heart, as well as to monitor the patient for the occurrence of aortic insufficiency. In some embodiments, the output of the remote accelerometer 30 is processed by the controller 20 to detect and/or measure heart rate. In some embodiments, the output of the remote accelerometer 30 is processed by the controller 20 using a suitable band-pass filter (e.g., 0.5 to 4.0 Hz) to isolate accelerations due to the heart rate. The accelerations due to the heart rate can be processed by the controller 20 to determine and track the heart rate.

The cardiac cycle timing of the patient is detected via monitoring of drive current supplied to the VAD 14, rotational speed of the rotor/impeller 140, flow rate of blood through the VAD 14, and/or pressure differential across the rotor/impeller 140. For example, during ventricular systole, variation in the ventricular pressure induces corresponding changes in the drive current supplied to the VAD 14 for a given rotational speed of the rotor/impeller 140. In some embodiments, the rotation speed of the rotor/impeller 140 is kept constant over one or more cardiac cycles to avoid inducing changes in the drive current due to changes in the rotational speed of the rotor/impeller 140. By monitoring the drive current supplied to the VAD 14, the cardiac cycle timing can be detected via detection of the time periods corresponding to ventricular systole. For example, in many embodiments the rotor drive current essentially follows a sinusoidal shape throughout the cardiac cycle. Peak flows (and drive current) occur at maximum left ventricle pressure, which is in the middle of systole. Minimum flows (min drive current) occur at the lowest left ventricle pressure, which is the start of diastole. In summary, the start of diastole can be detected by detecting the minimum drive current. Peak systole can be detected by detecting maximum drive current. Start of systole can be detected by detecting a sudden change in drive current slope (dI/dt) at the end of diastole.

In act 78, the output of the remote accelerometer 30 is processed by the controller 20 to track the patient's respiration. The patient's respiration rate and the diaphragm contraction amplitude can be determined by the controller 20 by processing the output of the remote accelerometer 30 using a suitable band-pass filter (e.g., approximately 0.2 to 1.0 Hz (12 to 60 breaths per minute)) to isolate accelerations due to respiration. The resulting accelerations due to respiration can then be processed by the controller 20 to determine corresponding respiration rate and diaphragm contraction amplitude. Frequency range and direction of movement can be used to isolate respiratory motion. In addition, accelerations due to respiration will typically have lower amplitudes (1 to 10 mg) and a regular pattern.

In act 80, the controller 20 monitors an activity level of the patient 12. The activity level of the patient 12 can be defined to be a suitable function of one or more of the respiration rate, the diaphragm contraction amplitude, the ventricle contraction amplitude, the heart rate, and/or the orientation of the patient 12.

In act 82, the controller 20 controls operation of the VAD 14 based on the patient's activity level and/or cardiac cycle timing. In some embodiments, the controller 20 controls the rotational rate of the rotor/impeller 140 so that the output of the VAD 14 is varied based on the measured activity level so as to provide increased support in response to an increase in the measured activity level and decreased support in response to a decrease in the activity level. In some embodiments, the controller 20 increases the average rotational speed of the rotor/impeller 140 to increase the output of the VAD 14 and decreases the average rotational speed to decrease the output of the VAD 14.

In some embodiments, the controller 20 controls operation of the VAD 14 to synchronize operation of the VAD 14 with the cardiac cycle timing. In addition to determining the cardiac cycle timing via processing of the output of the remote accelerometer 30, the controller 20 can determine the cardiac cycle timing based on operating parameters of the VAD 14, such as current, pump speed, and/or flow rate.

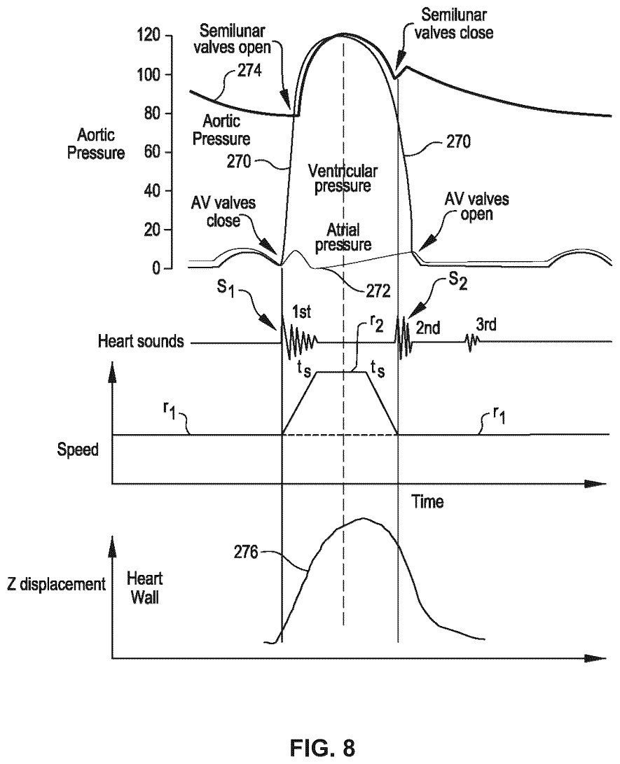

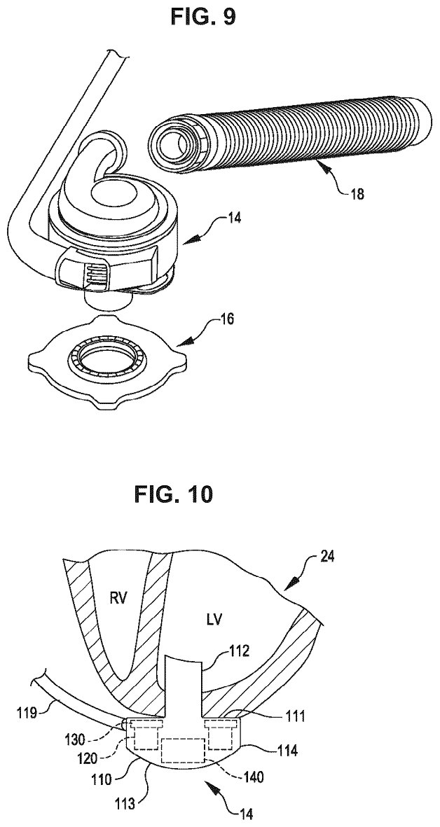

FIG. 8 illustrates synchronization of speed variation of the VAD 14 with ventricular systole based on heart sounds, ventricular wall motion, and/or pump operating parameters, in accordance with many embodiments. As described herein, the VAD 14 can include a VAD accelerometer 210 that measures accelerations of the VAD 14 that can be processed by the controller 20 to track ventricular wall motion. At the beginning of the cardiac cycle, both the atria and ventricles are relaxed (diastole). Blood flows into the atriums and into the ventricles from the atriums. Contraction of the atria (atrial systole) pumps additional blood from the atriums into the ventricles. Atrial systole ends prior to ventricular systole. During ventricular systole, each of the ventricular pressures 270 (only one shown for clarity) increases over the respective atrial pressure 272 (only one shown for clarity) thereby causing the respective atrial valve to close. The closing of the atrial valves generates the first heart sound (S1).

Further contraction of the respective ventricle increases the ventricular pressure 270 to above the respective output blood vessel pressure (e.g., aortic pressure 274), thereby causing the respective semilunar valve to open and blood to flow out of the ventricle. Ventricular relaxation (ventricular diastole) follows ventricular systole. As the ventricles relax, each of the ventricular pressures 270 drops below the respective output blood vessel pressure, thereby causing the respective semilunar valve to close. The closure of the semilunar valves generates the second heart sound (S2). Further relaxation of the ventricles decreases each of the ventricular pressures below the respective atrial pressure (e.g., left atrial pressure 272), thereby causing the atrial valves to open.

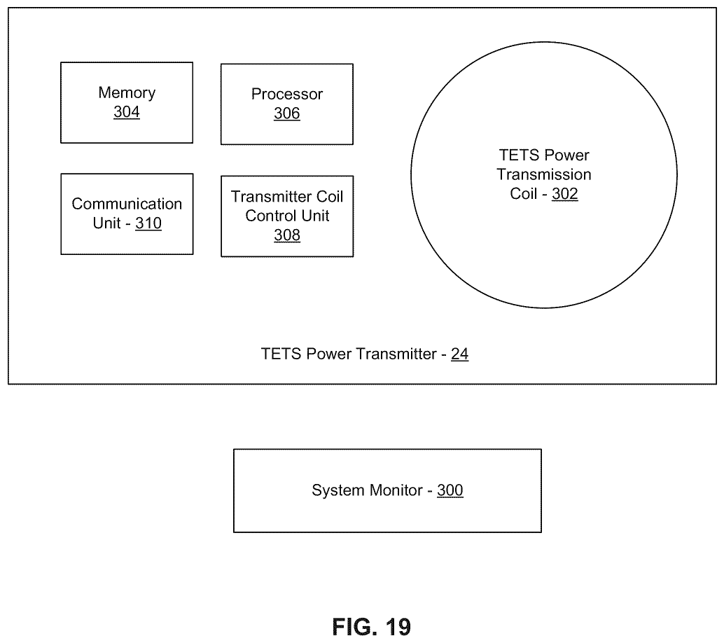

Heart wall motion 276 during contraction of the ventricles during ventricular systole induces accelerations of the VAD 14 that are measured by the accelerometer 210. In the illustrated embodiment, the heart wall motion induced acceleration of the VAD 14 is primarily reflected in the Z-axis acceleration 240 (example shown in FIG. 16) measured by the accelerometer 210. Accordingly, the Z-axis displacement 266 (example shown in FIG. 19) can be processed to monitor the heart wall motion 276 to measure timing and strength of each ventricular systole, and thereby indicating cardiac cycle timing. Detection of the heart sounds (S1 and S2) can also be used to determine the cardiac cycle timing, either alone or in combination with the timing of ventricular systole determined via assessment of the heart wall motion 276.

The motion of the ventricular heart wall can be monitored by processing output of the accelerometer 210 using a suitable band-pass filter (e.g., primary left ventricle wall motion range should be 0.5 to 3 Hz (30 to 180 BPM)) to isolate accelerations due to the ventricular heart wall motion. The heart wall motion can be processed to determine cardiac cycle timing, ventricle contractile strength, and ventricle contractile efficiency. Direction (z-axis, in line with the inflow cannula), frequency range, and timing regularity can be used to isolate LV wall motion. Transitions from periods of low accelerations to high accelerations (or changes in acceleration, jerk) can be used to indicate the start of a cardiac cycle (start of systole). Maximum acceleration can be used to estimate contractile strength. Contractile strength combined with min/mean/max flow through the pump can then be used to estimate contractile efficiency. The ventricle contractile strength and ventricle contractile efficiency can be monitored to monitor health of the patient's heart (e.g., detect signs of recovery or weakening). The heart wall motion can also be monitored to detect arrhythmia. Irregular cardiac cycle timing periods (start of systole) can be used to detect arrhythmia. In addition, heart rates above and below a normal range can be used to detect arrhythmia.

In act 82, the VAD 14 can be operated so as to vary output of the VAD 14 in synchronization with the cardiac cycle timing. For example, the VAD 14 can be operated to pump blood at a greater rate during ventricular systole than pumped by the VAD 14 during the rest of the cardiac cycle. The rotation speed of the rotor/impeller 140 can be varied to vary the rate that the VAD 14 pumps blood. Any suitable variation of the output of the VAD 14 can be used. For example, as shown in FIG. 8, the rotational speed of the rotor/impeller 140 can be varied during ventricular systole to increase the output of the VAD 14 during ventricular systole. In the illustrated example, the rotational speed of the rotor/impeller 140 is varied during ventricular systole (i.e., increased from a first rotational speed (r1) to a second rotational speed (r2), maintained at the second rotational speed (r2) for a period of time, and then reduced back down to the first rotational speed (r1)).

Any suitable approach can be used to control timing of the variation in output of the VAD 14. For example, cardiac cycle timing observed during one or more previous cardiac cycles can be used to control timing of the variation in output of the VAD 14 during a current cardiac cycle. As another example, when sufficiently fast processing is utilized, cardiac cycle timing for a target cardiac cycle can be used to control timing of the variation in output of the VAD 14 during the target cardiac cycle.

In act 84, the controller 20 processes the remote accelerations to monitor for occurrences of operational problems of the VAD 14. The accelerations measured by the remote accelerometer 30 may include accelerations induced via operation of the VAD 14. The induced accelerations can include accelerations induced via vibrations of the rotor/impeller 140, speed of the rotor/impeller 140, change in speed of the rotor/impeller 140, and/or mass/balance of the rotor/impeller 140. The accelerations measured by the remote accelerometer 30 can also be induced as the result of ingestion, by the VAD 14, of an object such as a blood clot. The accelerations measured by the remove accelerometer 32 can also be induced by a suction event, which can occur when the VAD 14 over-extracts blood from the ventricle. The output of the remote accelerometer 30 can be processed using a suitable band-pass filter (e.g., 0.5 to 3.0 Hz) to isolate accelerations induced via operation of the VAD 14.

The accelerations induced via operation of the VAD 14 can be processed to monitor for excessive rotor vibration, ingestion of an object, and/or the occurrence of a suction event. For example, a suitable band-pass filter can be applied around the operating rotor speed (50-150 Hz) and potentially the subsequent harmonics. Changes in vibration amplitude (specifically increases) of the resulting filtered accelerations can be indicative of a rotor imbalance caused by either an ingested thrombus or thrombus forming on the rotor. Suction events can be detected via the combined occurrence of three events: (1) changes in LV wall motion (as measured by the VAD accelerometer 210), (2) low average flow through the pump (which can be detected via rotor drive current), and (3) low minimal flow fluctuations (which can be detected via the occurrence of small changes in drive current).

In act 86, the controller 20 generates patient monitoring data. The generated patient monitoring data can be any suitable combination of the orientation(s) of the patient, the patient's cardiac activity, and the patient's respiration. The controller 20 outputs the patient monitoring data (act 88) and the VAD monitoring data (act 90) via the communication unit 58.

In act 92, the controller 20 periodically operates the haptic unit 60 for a brief period of time and processes the output of the remote accelerometer 30 to determine whether the haptic unit 60 actually generated accelerations of the remote accelerometer 30 consistent with proper operation of the haptic unit 60. If the controller 20 determines that the output of the remote accelerometer 30 is not consistent with proper operation of the haptic unit 60, the controller 20 outputs a haptic unit failure alarm via the communication unit 58 to notify the patient and/or a health care professional of the failed status of the haptic unit 60.

### Example VAD Configuration

With reference to FIG. 10 and FIG. 11, the VAD 14 has a circular shaped housing 110 and is shown implanted within the patient 12 with a first face 111 of the housing 110 positioned against the patient's heart 32 and a second face 113 of the housing 110 facing away from the heart 32. The first face 111 of the housing 110 includes an inlet cannula 112 extending into the left ventricle LV of the heart 32. The second face 113 of the housing 110 has a chamfered edge 114 to avoid irritating other tissue that may come into contact with the VAD 14, such as the patient's diaphragm. To construct the illustrated shape of the puck-shaped housing 110 in a compact form, a stator 120 and electronics 130 of the VAD 14 are positioned on the inflow side of the housing toward first face 111, and a rotor/impeller 140 of the VAD 14 is positioned along the second face 113. This positioning of the stator 120, electronics 130, and rotor/impeller 140 permits the edge 114 to be chamfered along the contour of the rotor/impeller 140, as illustrated in at least FIG. 10 and FIG. 11, for example.

Referring to FIG. 11, the VAD 14 includes a dividing wall 115 within the housing 110 defining a blood flow conduit 103. The blood flow conduit 103 extends from an inlet opening 101 of the inlet cannula 112 through the stator 120 to an outlet opening 105 defined by the housing 110. The rotor/impeller 140 is positioned within the blood flow conduit 103. The stator 120 is disposed circumferentially about a first portion 140a of the rotor/impeller 140, for example about a permanent magnet 141. The stator 120 is also positioned relative to the rotor/impeller 140 such that, in use, blood flows within the blood flow conduit 103 through the stator 120 before reaching the rotor/impeller 140. The permanent magnet 141 has a permanent magnetic north pole N and a permanent magnetic south pole S for combined active and passive magnetic levitation of the rotor/impeller 140 and for rotation of the rotor/impeller 140. The rotor/impeller 140 also has a second portion 140b that includes impeller blades 143. The impeller blades 143 are located within a volute 107 of the blood flow conduit such that the impeller blades 143 are located proximate to the second face 113 of the housing 110.

The puck-shaped housing 110 further includes a peripheral wall 116 that extends between the first face 111 and a removable cap 118. As illustrated, the peripheral wall 116 is formed as a hollow circular cylinder having a width W between opposing portions of the peripheral wall 116. The housing 110 also has a thickness T between the first face 111 and the second face 113 that is less than the width W. The thickness T is from about 0.5 inches to about 1.5 inches, and the width W is from about 1 inch to about 4 inches. For example, the width W can be approximately 2 inches, and the thickness T can be approximately 1 inch.

The peripheral wall 116 encloses an internal compartment 117 that surrounds the dividing wall 115 and the blood flow conduit 103, with the stator 120 and the electronics 130 disposed in the internal compartment 117 about the dividing wall 115. The removable cap 118 includes the second face 113, the chamfered edge 114, and defines the outlet opening 105. The cap 118 can be threadedly engaged with the peripheral wall 116 to seal the cap 118 in engagement with the peripheral wall 116. The cap 118 includes an inner surface 118a of the cap 118 that defines the volute 107 that is in fluid communication with the outlet opening 105.

Within the internal compartment 117, the electronics 130 are positioned adjacent to the first face 111 and the stator 120 is positioned adjacent to the electronics 130 on an opposite side of the electronics 130 from the first face 111. The electronics 130 include circuit boards 131 and various components carried on the circuit boards 131 to control the operation of the VAD 14 (e.g., magnetic levitation and/or drive of the rotor) by controlling the electrical supply to the stator 120. The housing 110 is configured to receive the circuit boards 131 within the internal compartment 117 generally parallel to the first face 111 for efficient use of the space within the internal compartment 117. The circuit boards also extend radially inward towards the dividing wall 115 and radially outward towards the peripheral wall 116. For example, the internal compartment 117 is generally sized no larger than necessary to accommodate the circuit boards 131, and space for heat dissipation, material expansion, potting materials, and/or other elements used in installing the circuit boards 131. Thus, the external shape of the housing 110 proximate the first face 111 generally fits the shape of the circuits boards 131 closely to provide external dimensions that are not much greater than the dimensions of the circuit boards 131.

With continued reference to FIG. 11, the stator 120 includes a back iron 121 and pole pieces 123a-123f arranged at intervals around the dividing wall 115. The back iron 121 extends around the dividing wall 115 and is formed as a generally flat disc of a ferromagnetic material, such as steel, in order to conduct magnetic flux. The back iron 121 is arranged beside the control electronics 130 and provides a base for the pole pieces 123a-123f.

Each of the pole piece 123a-123f is L-shaped and has a drive coil 125 for generating an electromagnetic field to rotate the rotor/impeller 140. For example, the pole piece 123a has a first leg 124a that contacts the back iron 121 and extends from the back iron 121 towards the second face 113. The pole piece 123a can also have a second leg 124b that extends from the first leg 124a through an opening of a circuit board 131 towards the dividing wall 115 proximate the location of the permanent magnet 141 of the rotor/impeller 140. In an aspect, each of the second legs 124b of the pole pieces 123a-123f is sticking through an opening of the circuit board 131. In an aspect, each of the first legs 124a of the pole pieces 123a-123f is sticking through an opening of the circuit board 131. In an aspect, the openings of the circuit board are enclosing the first legs 124a of the pole pieces 123a-123f.

In a general aspect, the VAD 14 can include one or more Hall sensors that may provide an output voltage, which is directly proportional to a strength of a magnetic field that is located in between at least one of the pole pieces 123a-123f and the permanent magnet 141, and the output voltage may provide feedback to the control electronics 130 of the VAD 14 to determine if the rotor/impeller 140 and/or the permanent magnet 141 is not at its intended position for the operation of the VAD 14. For example, a position of the rotor/impeller 140 and/or the permanent magnet 141 can be adjusted, e.g., the rotor/impeller 140 or the permanent magnet 141 may be pushed or pulled towards a center of the blood flow conduit 103 or towards a center of the stator 120.

Each of the pole pieces 123a-123f also has a levitation coil 127 for generating an electromagnetic field to control the radial position of the rotor/impeller 140. Each of the drive coils 125 and the levitation coils 127 includes multiple windings of a conductor around the pole pieces 123a-123f. Particularly, each of the drive coils 125 is wound around two adjacent ones of the pole pieces 123, such as pole pieces 123d and 123e, and each levitation coil 127 is wound around a single pole piece. The drive coils 125 and the levitation coils 127 are wound around the first legs of the pole pieces 123, and magnetic flux generated by passing electrical current though the coils 125 and 127 during use is conducted through the first legs and the second legs of the pole pieces 123 and the back iron 121. The drive coils 125 and the levitation coils 127 of the stator 120 are arranged in opposing pairs and are controlled to drive the rotor and to radially levitate the rotor/impeller 140 by generating electromagnetic fields that interact with the permanent magnetic poles S and N of the permanent magnet 141.

Because the stator 120 includes both the drive coils 125 and the levitation coils 127, only a single stator is needed to levitate the rotor/impeller 140 using only passive and active magnetic forces. The permanent magnet 141 in this configuration has only one magnetic moment and is formed from a monolithic permanent magnetic body 141. For example, the stator 120 can be controlled as discussed in U.S. Pat. No. 6,351,048, the entire contents of which are incorporated herein by reference for all purposes. Further related patents, namely U.S. Pat. Nos. 5,708,346, 6,053,705, 6,100,618, 6,222,290, 6,249,067, 6,278,251, 6,351,048, 6,355,998, 6,634,224, 6,879,074, and 7,112,903, all of which are incorporated herein by reference for all purposes in their entirety.

The rotor/impeller 140 is arranged within the housing 110 such that its permanent magnet 141 is located upstream of impeller blades in a location closer to the inlet opening 101. The permanent magnet 141 is received within the blood flow conduit 103 proximate the second legs 124b of the pole pieces 123 to provide the passive axial centering force though interaction of the permanent magnet 141 and ferromagnetic material of the pole pieces 123. The permanent magnet 141 of the rotor/impeller 140 and the dividing wall 115 form a gap 108 between the permanent magnet 141 and the dividing wall 115 when the rotor/impeller 140 is centered within the dividing wall 115. The gap 108 may be from about 0.2 millimeters to about 2 millimeters. For example, the gap 108 can be approximately 1 millimeter. The north permanent magnetic pole N and the south permanent magnetic pole S of the permanent magnet 141 provide a permanent magnetic attractive force between the rotor/impeller 140 and the stator 120 that acts as a passive axial centering force that tends to maintain the rotor/impeller 140 generally centered within the stator 120 and tends to resist the rotor/impeller 140 from moving towards the first face 111 or towards the second face 113. When the gap 108 is smaller, the magnetic attractive force between the permanent magnet 141 and the stator 120 is greater, and the gap 108 is sized to allow the permanent magnet 141 to provide the passive magnetic axial centering force having a magnitude that is adequate to limit the rotor/impeller 140 from contacting the dividing wall 115 or the inner surface 118a of the cap 118. The rotor/impeller 140 also includes a shroud 145 that covers the ends of the impeller blades 143 facing the second face 113 that assists in directing blood flow into the volute 107. The shroud 145 and the inner surface 118a of the cap 118 form a gap 109 between the shroud 145 and the inner surface 118a when the rotor/impeller 140 is levitated by the stator 120. The gap 109 is from about 0.2 millimeters to about 2 millimeters. For example, the gap 109 is approximately 1 millimeter.

As blood flows through the blood flow conduit 103, blood flows through a central aperture 141a formed through the permanent magnet 141. Blood also flows through the gap 108 between the rotor/impeller 140 and the dividing wall 115 and through the gap 109 between the shroud 145 and the inner surface 108a of the cap 118. The gaps 108 and 109 are large enough to allow adequate blood flow to limit clot formation that may occur if the blood is allowed to become stagnant. The gaps 108 and 109 are also large enough to limit pressure forces on the blood cells such that the blood is not damaged when flowing through the VAD 14. As a result of the size of the gaps 108 and 109 limiting pressure forces on the blood cells, the gaps 108 and 109 are too large to provide a meaningful hydrodynamic suspension effect. That is to say, the blood does not act as a bearing within the gaps 108 and 109, and the rotor is only magnetically levitated. In various embodiments, the gaps 108 and 109 are sized and dimensioned so the blood flowing through the gaps forms a film that provides a hydrodynamic suspension effect. In this manner, the rotor can be suspended by magnetic forces, hydrodynamic forces, or both.

Because the rotor/impeller 140 is radially suspended by active control of the levitation coils 127 as discussed above, and because the rotor/impeller 140 is axially suspended by passive interaction of the permanent magnet 141 and the stator 120, no magnetic field generating rotor levitation components are needed proximate the second face 113. The incorporation of all the components for rotor levitation in the stator 120 (i.e., the levitation coils 127 and the pole pieces 123) allows the cap 118 to be contoured to the shape of the impeller blades 143 and the volute 107. Additionally, incorporation of all the rotor levitation components in the stator 120 eliminates the need for electrical connectors extending from the compartment 117 to the cap 118, which allows the cap to be easily installed and/or removed and eliminates potential sources of pump failure.

In use, the drive coils 125 of the stator 120 generates electromagnetic fields through the pole pieces 123 that selectively attract and repel the magnetic north pole N and the magnetic south pole S of the rotor/impeller 140 to cause the rotor/impeller 140 to rotate within stator 120. For example, the one or more Hall sensors may sense a current position of the rotor/impeller 140 and/or the permanent magnet 141, wherein the output voltage of the one or more Hall sensors may be used to selectively attract and repel the magnetic north pole N and the magnetic south pole S of the rotor/impeller 140 to cause the rotor/impeller 140 to rotate within stator 120. As the rotor/impeller 140 rotates, the impeller blades 143 force blood into the volute 107 such that blood is forced out of the outlet opening 105. Additionally, the rotor draws blood into VAD 14 through the inlet opening 101. As blood is drawn into the blood pump by rotation of the impeller blades 143 of the rotor/impeller 140, the blood flows through the inlet opening 101 and flows through the control electronics 130 and the stator 120 toward the rotor/impeller 140. Blood flows through the aperture 141a of the permanent magnet 141 and between the impeller blades 143, the shroud 145, and the permanent magnet 141, and into the volute 107. Blood also flows around the rotor/impeller 140, through the gap 108 and through the gap 109 between the shroud 145 and the inner surface 118a of the cap 118. The blood exits the volute 107 through the outlet opening 105, which may be coupled to an outflow cannula.

FIG. 12 shows a Hall Sensor assembly 200 for the VAD 14, in accordance with many embodiments. The Hall Sensor assembly 200 includes a printed circuit board assembly (PCBA) 202 and six individual Hall Effect sensors 208 supported by the printed circuit board 202. The Hall Effect sensors 208 are configured to transduce a position of the rotor/impeller 140 of the VAD 14. In the illustrated embodiment, the Hall Effect sensors 208 are supported so as to be standing orthogonally relative to the PCBA 202 and a longest edge of each of the Hall Effect sensors 208 is aligned to possess an orthogonal component with respect to the surface of the PCBA 202. Each of the Hall Effect sensors 208 generates an output voltage, which is directly proportional to a strength of a magnetic field that is located in between at least one of the pole pieces 123a-123f and the permanent magnet 141. The voltage output by each of the Hall Effect sensors 208 is received by the control electronics 130, which processes the sensor output voltages to determine the position and orientation of the rotor/impeller 140. The determined position and orientation of the rotor/impeller 140 is used to determine if the rotor/impeller 140 is not at its intended position for the operation of the VAD 14. For example, a position of the rotor/impeller 140 and/or the permanent magnet 141 may be adjusted, for example, the rotor/impeller 140 or the permanent magnet 141 may be pushed or pulled towards a center of the blood flow conduit 103 or towards a center of the stator 120. The determined position of the rotor/impeller 140 can also be used to determine rotor eccentricity or a target rotor eccentricity, which can be used as described in U.S. Pat. No. 9,901,666, all of which is incorporated herein by reference for all purposes in its entirety, to estimate flow rate of blood pumped by the VAD 14.

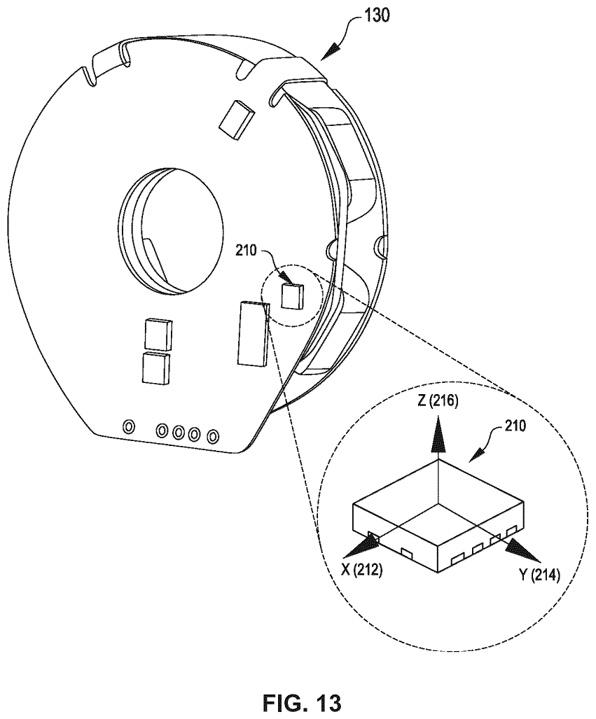

FIG. 13 is a heart-side view of the control electronics 130 showing an accelerometer 210 included in the control electronics 130, in accordance with many embodiments. In the many embodiments, the accelerometer 210 is a three-axis accelerometer that measures accelerations experienced by the control electronics 130 (and thereby the VAD 14) in three orthogonal axes (i.e., an X-axis 212, a Y-axis 214, and a Z-axis 216). In the illustrated embodiment, the X-axis 212 and the Y-axis 214 are each oriented orthogonal to an axis of rotation of the rotor/impeller 140, and the Z-axis 216 is parallel to the axis of rotation of the rotor/impeller 140.

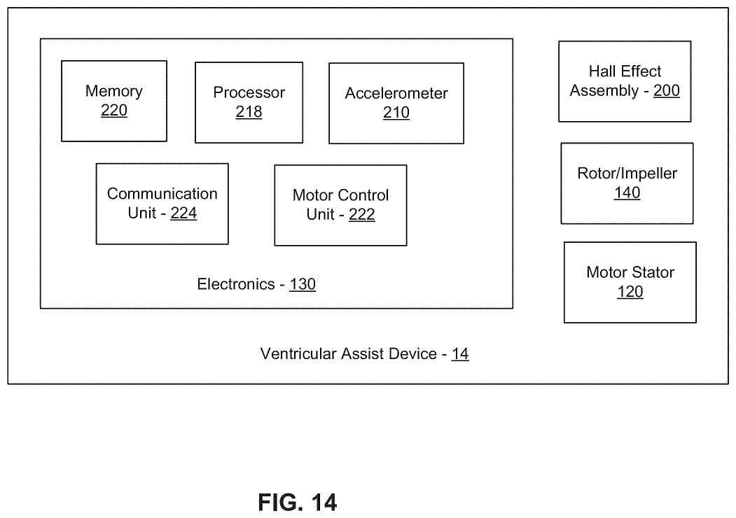

FIG. 14 is a schematic diagram of the VAD 14. The VAD 14 includes the control electronics 130, the Hall Effect Sensor assembly 200, the motor stator 120, the rotor/impeller 140. In the illustrated embodiment, the control electronics include a processor 218, a memory device 220 (which can include read-only memory and/or random access-memory), the accelerometer 210, a motor control unit 222, and a communication unit 224. In some embodiments, the memory device 220 stores one or more software applications that are executable by the processor 218 for various functions. For example, the one or more software applications can effectuate control the motor control unit 222 to effectuate radial levitation and rotational drive of the rotor/impeller 140 during operation. In some embodiments, the one or more programs effectuate processing of output from the accelerometer 210 and/or operational parameters for the VAD 14 (e.g., drive current, rotational speed, flow rate, pressure differential across the impeller) as described herein to detect and/or measure patient physiological events and/or activity (e.g., patient orientation, patient activity level, heart wall motion, heart sounds, heart rate, respiratory rate, diaphragm contraction, cardiac cycle timing). The one or more programs can effectuate control of the motor control unit 222 to synchronize variation in output of the VAD 14 with the patient's cardiac cycle timing as described herein. For example, the output of the VAD 14 can be increased over a period of time during ventricular systole so as to augment pumping of blood that occurs via contraction of the ventricle, thereby reducing the associated load on the ventricle. The one or more programs can effectuate control of the motor control unit 222 to vary output of the VAD 14 based on patient activity level. For example, in many embodiments, the output of the VAD 14 is increased in response to increased patient activity and decreased in response to decreased patient activity. The one or more programs can also be used to effectuate processing of the output from the accelerometer 210 and/or the operational parameters for the VAD 14 to generate patient monitoring data and/or VAD monitoring data as described herein. The communication unit 224 provides for wired and/or wireless communication between the VAD 14 and the controller 20. In some embodiments, the motor control unit 222 is included in the VAD 14. In other embodiments, the motor control unit 222 is included in the controller 20.

### Example Measured Accelerations

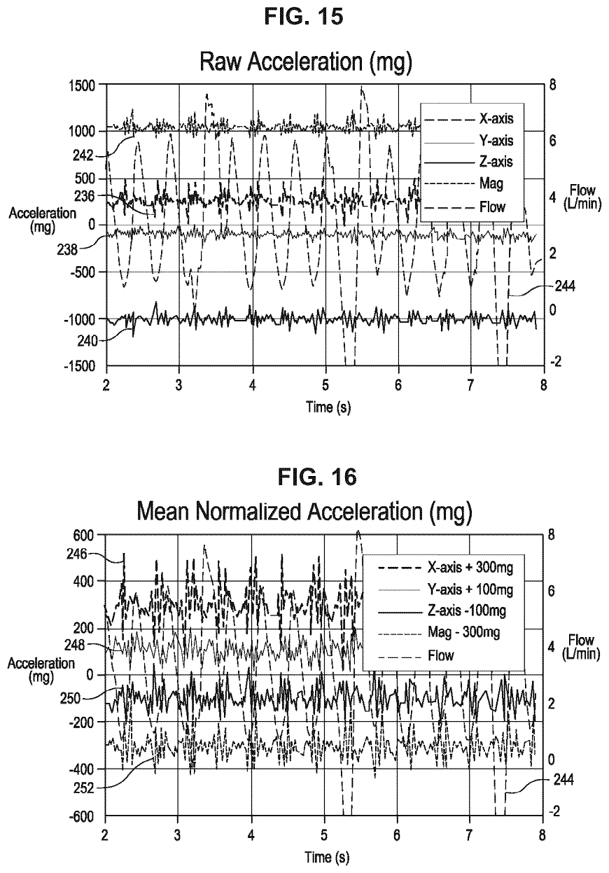

FIG. 15 is a plot of raw accelerations of the VAD 14 measured by the three-axis accelerometer 210 during an animal study. The raw accelerations shown include X-axis acceleration 236, Y-axis acceleration 238, Z-axis acceleration 240, and a magnitude 242 of the raw acceleration. FIG. 15 also shows a flow rate 244 of the VAD 14 during the measurement of the raw accelerations.

FIG. 16 is a plot of mean normalized accelerations of the VAD 14 generated from the raw accelerations of FIG. 15. The mean normalized accelerations shown include X-axis mean normalized acceleration 246, Y-axis mean normalized acceleration 248, Z-axis mean normalized acceleration 250, and a magnitude 252 of the mean normalized acceleration. Each of the mean accelerations was produced by subtracting the corresponding average acceleration over the entire sample period from the corresponding raw acceleration plot (so that the resulting average is zero). FIG. 16 also shows the flow rate 244 of the VAD 14 during the measurement of the raw accelerations. To enhance legibility of FIG. 16, a constant acceleration offset has been combined with each of the acceleration components (i.e., 300 mg added to the X-axis mean normalized acceleration 246, 100 mg has been added to the Y-axis mean normalized acceleration 248, 100 mg has been subtracted from the Z-axis mean normalized acceleration 250, and 300 mg has been subtracted from the magnitude 252 of the total mean normalized acceleration) so as to separate the plotted components.

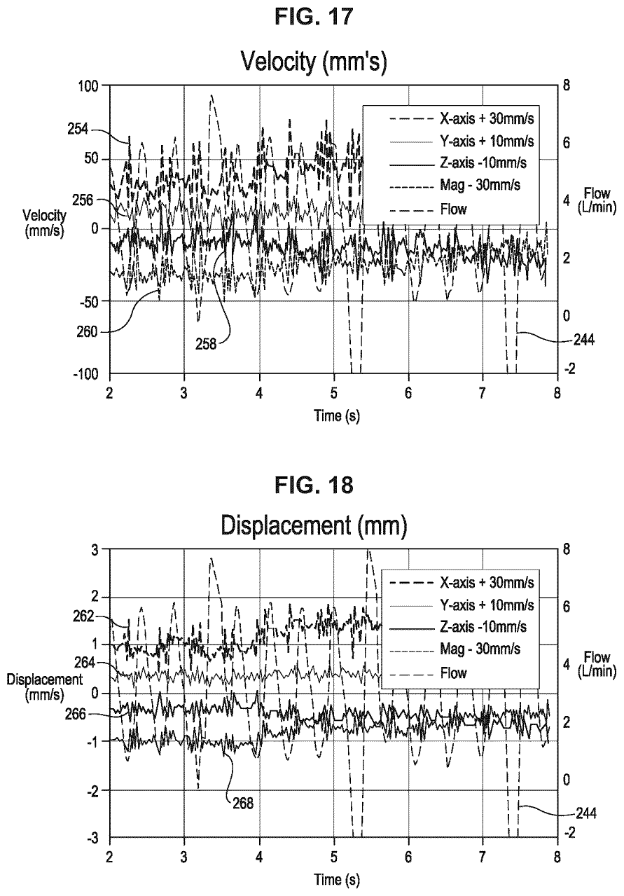

FIG. 17 is a plot of velocities of the VAD 14 generated via integration of the mean normalized accelerations of FIG. 16. The output from at least three accelerometers (or one accelerometer and one gyroscope) can be integrated to determine the corresponding three-dimensional velocity. With fewer accelerometers, significant amounts of rotational motion may induce significant levels of error in the resulting three-dimensional velocity. While one accelerometer can provide reasonable estimates of the velocity if the rotational motions are insignificant, rotational motions of a VAD may be significant. For example, if the beating of the heart rocks the accelerometer back and forth in a motion that includes rotation, an error due to centripetal acceleration may accumulate. While the error may be quite small for a time, the error may grow in size over time. In many embodiments, acceleration due to gravity is filtered out prior to integrating the accelerations to determine the three-dimensional velocity. The velocities shown include X-axis velocity 254, Y-axis velocity 256, Z-axis velocity 258, and total velocity 260. FIG. 17 also shows the flow rate 244 of the VAD 14 during the measurement of the raw accelerations. To enhance legibility of FIG. 17, a constant velocity offset has been combined with each of the velocity components (i.e., 30 mm/sec added to the X-axis velocity 254, 10 mm/sec has been added to the Y-axis velocity 256, 10 mm/sec has been subtracted from the Z-axis velocity 258, and 30 mm/sec has been subtracted from the total velocity 260) so as to separate the plotted components.

FIG. 18 is a plot of displacements of the VAD 14 generated via integration of the velocities of FIG. 17. The displacements shown include X-axis displacement 262, Y-axis displacement 264, Z-axis displacement 266, and total displacement 268. FIG. 18 also shows the flow rate 244 of the VAD 14 during the measurement of the raw accelerations. To enhance legibility of FIG. 18, a constant displacement offset has been combined with each of the displacement components (i.e., 1 mm added to the X-axis displacement 262, 0.3 mm has been added to the Y-axis displacement 264, 0.3 mm has been subtracted from the Z-axis displacement 266, and 30 mm/sec has been subtracted from the total displacement 268) so as to separate the plotted components.

### External System Components

FIG. 19 is a schematic diagram of external components of the blood circulation assist system 10. The external components of the system 10 include the TETS power transmitter 24 and the system monitor 300. The TETS power transmitter 24 includes a TETS power transmission coil 302, a memory 304, a processor 306, a power transmission coil control unit 308, and a communication unit 310. The power transmission coil control unit 308 is configured to control operation of the TETS power transmission coil 302 under the control of the processor 306. The memory 304 stores instructions that are executable by the processor 306 to control the operation of the TETS power transmission coil control unit. The TETS power transmitter 24 can include a housing that defines an internal volume in which the TETS power transmission coil 302 is disposed. In some embodiments, TETS power transmitter 24 is deformable and/or flexible so to be at least partially conformable with patient anatomy when placed on the patient's body for transmitting power to the TETS power receiver 22.

In many embodiments, the TETS power transmitter 24 is configured to be coupled to an electric power source 212 such as an electrical wall outlet or other suitable external power sources. The TETS power transmission coil 302 can have any suitable resonant frequency. For example, the resonant frequency of the TETS power transmission coil 302 can be in a range of 100 kHz to 10 MHz, or in a range of 100 kHz to 20 MHz.

In many embodiments, the external system monitor 300 is configured to monitor operation of the implanted components of the system 10. The external system monitor 300 can receive the patient monitoring data and the VAD monitoring data from the controller 20. The external system monitor 300 can received any alarms output by the implanted controller 20, including the haptic unit failed status alarm and any alarm generated as a result of occurrence of a VAD operational problem, such as a suction event, a VAD thrombus vent, and instability of the rotor/impeller 140. In some embodiments, the external system monitor 300 is operable to download software updates to the controller 20, the VAD 14, and/or the TETS power receiver 22.

### EXAMPLE EMBODIMENTS

Example 1 is a blood circulation assist system that includes a ventricular assist

device (VAD), a transcutaneous energy transfer system (TETS) power receiver, a controller, and a remote accelerometer. The VAD includes an inlet, an outlet, an impeller, and a motor stator operable to rotate the impeller to pump a blood flow The inlet is configured for coupling with a ventricle of a patient to receive the blood flow from the ventricle. The outlet is configured for coupling with a blood vessel of the patient to transfer the blood flow to the blood vessel. The TETS power receiver is configured to be implanted and receive energy transmitted by an external TETS transmitter. The controller is configured to be implanted, process a remote accelerometer output, and control a rotation speed of the impeller based on the remote accelerometer output. The remote accelerometer is configured to generate the remote accelerometer output. In some embodiments, the TETS power receiver includes the remote accelerometer and the remote accelerometer output is indicative of accelerations of the TETS power receiver. In some embodiments, the controller includes the remote accelerometer and the remote accelerometer output is indicative of accelerations of the controller.

Example 2 is the blood circulation assist system of claim 1, wherein the TETS power receiver includes the remote accelerometer.

Example 3 is the blood circulation assist system of claim 2, wherein the TETS power receiver includes a TETS power receiver coil and a TETS power receiver housing that encloses the TETS power receiver coil and includes an outer surface of the TETS power receiver and the remote accelerometer is mounted to an inner surface of the TETS power receiver housing.

Example 4 is the blood circulation assist system of claim 3, wherein the TETS power receiver housing includes a titanium panel that includes the inner surface of the TETS power receiver housing and the outer surface of the TETS power receiver.

Example 5 is the blood circulation assist system of claim 2, wherein the TETS power receiver includes a TETS power receiver printed circuit board assembly (PCBA) and the remote accelerometer is mounted to the TETS power receiver PCBA.

Example 6 is the blood circulation assist system of claim 2, further including a TETS power receiver connection cable that connects the TETS power receiver to the controller.

Example 7 is the blood circulation assist system of claim 6, wherein the TETS power receiver is configured for implantation in a pectoral region of the patient.

Example 8 is the blood circulation assist system of claim 6, further including controller connection cable that connects the controller to the VAD.

Example 9 is the blood circulation assist system of claim 6, wherein the controller is configured for implantation in an abdominal wall region of the patient.

Example 10 is the blood circulation assist system of any one of example 1 through example 9, wherein the controller is configured to process the remote accelerometer output to determine a heart rate of the patient and control the rotation speed of the impeller based on the heart rate.

Example 11 is the blood circulation assist system of any one of example 1 through example 9, wherein the controller is configured to process the remote accelerometer output to determine a ventricular contraction magnitude of the patient.

Example 12 is the blood circulation assist system of any one of example 1 through example 9, wherein the controller is configured to process the remote accelerometer output to determine a cardiac output magnitude of the patient.

Example 13 is the blood circulation assist system of any one of example 1 through example 9, wherein the controller is configured to process the remote accelerometer output to determine a valve opening timing of the patient.

Example 14 is the blood circulation assist system of any one of example 1 through example 9, wherein the controller is configured to process the remote accelerometer output to monitor for a valve disorder of the patient.

Example 15 is the blood circulation assist system of any one of example 1 through example 9, wherein the controller is configured to process the remote accelerometer output to determine a respiration rate of the patient.

Example 16 is the blood circulation assist system of any one of example 1 through example 9, wherein the controller is configured to process the remote accelerometer output to monitor for an occurrence of pump thrombosis in the VAD.

Example 17 is the blood circulation assist system of any one of example 1 through example 9, wherein the controller is configured to process the remote accelerometer output to monitor for an occurrence of an occlusion in the VAD.

Example 18 is the blood circulation assist system of any one of example 1 through example 9, wherein the controller is configured to process the remote accelerometer output to monitor for an occurrence of an instability of the impeller.

Example 19 is the blood circulation assist system of any one of example 1 through example 9, wherein the controller is configured to process the remote accelerometer output to monitor an orientation of the patient.

Example 20 is the blood circulation assist system of any one of example 1 through example 9, wherein the controller is configured to process the remote accelerometer output to determine when the patient is prone.

Example 21 is the blood circulation assist system of any one of example 1 through example 9, wherein the controller is configured to process the remote accelerometer output to determine when the patient is supine.

Example 22 is the blood circulation assist system of example 21, wherein the controller is configured to process the remote accelerometer output to determine an angle of recline when the patient is supine.

Example 23 is the blood circulation assist system of any one of example 1 through example 9, wherein the controller is configured to process the remote accelerometer output to determine when the patient is sitting.

Example 24 is the blood circulation assist system of any one of example 1 through example 9, wherein the controller is configured to process the remote accelerometer output to determine when the patient is standing.

Example 25 is the blood circulation assist system of any one of example 1 through example 9, wherein the controller is configured to process the remote accelerometer output to monitor for a fall of the patient.

Example 26 is the blood circulation assist system of any one of example 1 through example 9, wherein the controller is configured to process the remote accelerometer output to monitor for a syncope of the patient.

Example 27 is the blood circulation assist system of any one of example 1 through example 9, wherein the controller is configured to process the remote accelerometer output to determine whether the patient is active or at rest.

Example 28 is the blood circulation assist system of any one of example 1 through example 9, wherein the controller is configured to process the remote accelerometer output to determine a wellness indicator for the patient.

Example 29 is the blood circulation assist system of any one of example 1 through example 9, wherein the controller is configured to process the remote accelerometer output to detect a cardiac cycle timing of the patient, the cardiac cycle timing includes a heart rate and a time of occurrence for each of one or more cardiac cycle events, and the controller is configured to vary the rotation speed of the impeller in sync with the cardiac cycle timing.

Example 30 is the blood circulation assist system of example 29, wherein the controller is configured to increase the rotation speed of the impeller to during ventricular systole.

Example 31 is the blood circulation assist system of example 30, wherein the controller is configured to process the remote accelerometer output to detect a time of occurrence of at least one heart sound and detect timing of ventricular systole based on the time of occurrence of the at least one heart sound.

Example 32 is the blood circulation assist system of example 31, wherein the at least one heart sound includes a sound of closure of at least one atrioventricular valve of the patient and/or a sound of closure of at least one semilunar valve of the patient.

Example 33 is the blood circulation assist system of example 29, wherein the controller is configured to vary the rotation speed of the impeller over a target cardiac cycle based on detected timing of one or more cardiac cycles that occur prior to the target cardiac cycle.

Example 34 is the blood circulation assist system of example 29, wherein the controller is configured to vary the rotation speed of the impeller over a target cardiac cycle based on detected timing of the target cardiac cycle.

Example 35 is the blood circulation assist system of example 29, wherein the controller is configured to process the remote accelerometer output to measure an activity level of the patient and control the rotation speed of the impeller based on the activity level.

Example 36 is the blood circulation assist system of example 35, wherein the controller is configured to process the remote accelerometer output to measure a respiration rate for the patient and/or a diaphragm contraction for the patient and base the activity level on the respiration rate and/or the diaphragm contraction.

Example 37 is the blood circulation assist system of any one of example 1 through example 9, wherein the controller is configured to process the remote accelerometer output to measure an activity level of the patient and control the rotation speed of the impeller based on the activity level.

Example 38 is the blood circulation assist system of example 37, wherein the controller is configured to process the remote accelerometer output to measure a respiration rate and/or a diaphragm contraction and base the activity level on the respiration rate and/or the diaphragm contraction.

Example 39 is the blood circulation assist system of any one of example 1 through example 9, wherein the remote accelerometer output is indicative of accelerations in three orthogonal directions.

Example 40 is a blood circulation assist system that includes a ventricular assist device (VAD), a transcutaneous energy transfer system (TETS) power receiver, a controller, and a remote accelerometer. The VAD includes a housing defining a blood flow channel, an inlet, an outlet, an impeller disposed within the blood flow channel, a motor stator, and a VAD accelerometer. The motor stator is operable to rotate the impeller to pump a blood flow in a patient. The inlet is configured for coupling with a ventricle of a heart to receive the blood flow from the ventricle. The outlet is configured for coupling with a blood vessel to transfer the blood flow to the blood vessel. The VAD accelerometer is configured to generate a VAD accelerometer output indicative of accelerations of the VAD. The TETS power receiver is configured to be implanted and receive energy transmitted by an external TETS transmitter. The controller is configured to be implanted, process the VAD accelerometer output and a remote accelerometer output, and control a rotation speed of the impeller based on at least one of the VAD accelerometer output and the remote accelerometer output. The remote accelerometer is configured to generate the remote accelerometer output. The TETS power receiver includes the remote accelerometer and the remote accelerometer output is indicative of accelerations of the TETS power receiver or the controller includes the remote accelerometer and the remote accelerometer output is indicative of accelerations of the controller.

Example 41 is the blood circulation assist system of example 40, wherein the VAD is configured to be mounted to a heart wall of the heart, the controller is configured to process the VAD accelerometer output to monitor motion of the heart wall to detect a cardiac cycle timing of the heart, and the controller is configured to control the rotation speed of the impeller based on the cardiac cycle timing.

Example 42 is the blood circulation assist system of example 41, wherein the TETS power receiver is configured for implantation in a pectoral region of the patient, the controller is configured to process the remote accelerometer output to determine a respiration rate of the patient, and the controller is configured to control the rotation speed of the impeller further based on the respiration rate.

Example 43 is the blood circulation assist system of any one of example 40 through example 42, wherein the VAD further includes control electronics configured to control drive currents supplied to the motor stator to rotate the impeller.

Example 44 is the blood circulation assist system of example 43, wherein the drive currents supplied to the motor stator are further used to magnetically levitate the impeller.

Example 45 is the blood circulation assist system of any one of example 40 through example 42, wherein the controller is configured to determine a posture of the patient based on the remote accelerometer output and the VAD accelerometer output.

Example 46 is a blood circulation assist system that includes a ventricular assist device (VAD), a controller, and a remote accelerometer. The VAD includes an inlet, an outlet, an impeller, and a motor stator operable to rotate the impeller to pump a blood flow.

The inlet is configured for coupling with a ventricle of a patient to receive the blood flow from the ventricle. The outlet is configured for coupling with a blood vessel of the patient to transfer the blood flow to the blood vessel. The controller is configured to process a remote accelerometer output indicative of accelerations of the patient at a remote accelerometer location and control a rotation speed of the impeller based on the remote accelerometer output. The remote accelerometer is configured to generate the remote accelerometer output. The remote accelerometer is configured to be implanted in the patient at the remote accelerometer location. The remote accelerometer location is separated from the VAD to isolate the remote accelerometer from noise generated by the VAD.

Example 47 is the blood circulation assist system of example 46, wherein the remote accelerometer is configured for implantation in a pectoral region of the patient.

Example 48 is the blood circulation assist system of example 46, wherein the remote accelerometer is configured for implantation in an abdominal wall region of the patient.

Example 49 is the blood circulation assist system of any one of example 46 through example 48, wherein the controller is configured to process the remote accelerometer output to determine a heart rate of the patient and control the rotation speed of the impeller based on the heart rate.

Example 50 is the blood circulation assist system of any one of example 46 through example 48, wherein the controller is configured to process the remote accelerometer output to determine a valve opening timing of the patient.

Example 51 is the blood circulation assist system of any one of example 46 through example 48, wherein the controller is configured to process the remote accelerometer output to monitor for a valve disorder of the patient.

Example 52 is the blood circulation assist system of any one of example 46 through example 48, wherein the controller is configured to process the remote accelerometer output to determine a respiration rate of the patient.

Example 53 is the blood circulation assist system of any one of example 46 through example 48, wherein the controller is configured to process the remote accelerometer output to monitor for an occurrence of pump thrombosis in the VAD.

Example 54 is the blood circulation assist system of any one of example 46 through example 48, wherein the controller is configured to process the remote accelerometer output to monitor for an occurrence of an occlusion in the VAD.

Example 55 is the blood circulation assist system of any one of example 46 through example 48, wherein the controller is configured to process the remote accelerometer output to monitor for an occurrence of an instability of the impeller.

Example 56 is the blood circulation assist system of any one of example 46 through example 48, wherein the controller is configured to process the remote accelerometer output to monitor an orientation of the patient.

Example 57 is the blood circulation assist system of any one of example 46 through example 48, wherein the controller is configured to process the remote accelerometer output to determine when the patient is prone.

Example 58 is the blood circulation assist system of any one of example 46 through example 48, wherein the controller is configured to process the remote accelerometer output to determine when the patient is supine.

Example 59 is the blood circulation assist system of example 58, wherein the controller is configured to process the remote accelerometer output to determine an angle of recline when the patient is supine.

Example 60 is the blood circulation assist system of any one of example 46 through example 48, wherein the controller is configured to process the remote accelerometer output to determine when the patient is sitting.

Example 61 is the blood circulation assist system of any one of example 46 through example 48, wherein the controller is configured to process the remote accelerometer output to determine when the patient is standing.

Example 62 is the blood circulation assist system of any one of example 46 through example 48, wherein the controller is configured to process the remote accelerometer output to monitor for a fall of the patient.

Example 63 is the blood circulation assist system of any one of example 46 through example 48, wherein the controller is configured to process the remote accelerometer output to monitor for a syncope of the patient.

Example 64 is the blood circulation assist system of any one of example 46 through example 48, wherein the controller is configured to process the remote accelerometer output to determine whether the patient is active or at rest.

Example 65 is the blood circulation assist system of any one of example 46 through example 48, wherein the controller is configured to process the remote accelerometer output to determine a wellness indicator for the patient.

Example 66 is the blood circulation assist system of any one of example 46 through example 48, wherein the controller is configured to process the remote accelerometer output to detect a cardiac cycle timing of the patient, the cardiac cycle timing includes a heart rate and a time of occurrence for each of one or more cardiac cycle events, and the controller is configured to vary the rotation speed of the impeller in sync with the cardiac cycle timing.

Example 67 is the blood circulation assist system of example 66, wherein the controller is configured to increase the rotation speed of the impeller to during ventricular systole.

Example 68 is the blood circulation assist system of example 67, wherein the controller is configured to process the remote accelerometer output to detect a time of occurrence of at least one heart sound and detect timing of ventricular systole based on the time of occurrence of the at least one heart sound.

Example 69 is the blood circulation assist system of example 68, wherein the at least one heart sound includes a sound of closure of at least one atrioventricular valve of the patient and/or a sound of closure of at least one semilunar valve of the patient.

Example 70 is the blood circulation assist system of example 66, wherein the controller is configured to vary the rotation speed of the impeller over a target cardiac cycle based on detected timing of one or more cardiac cycles that occur prior to the target cardiac cycle.

Example 71 is the blood circulation assist system of example 66, wherein the controller is configured to vary the rotation speed of the impeller over a target cardiac cycle based on detected timing of the target cardiac cycle.

Example 72 is the blood circulation assist system of example 66, wherein the

controller is configured to process the remote accelerometer output to measure an activity level of the patient and control the rotation speed of the impeller based on the activity level.

Example 73 is the blood circulation assist system of example 72, wherein the controller is configured to process the remote accelerometer output to measure a respiration rate for the patient and/or a diaphragm contraction for the patient and base the activity level on the respiration rate and/or the diaphragm contraction.

Example 74 is the blood circulation assist system of any one of example 46 through example 48, wherein the controller is configured to process the remote accelerometer output to measure an activity level of the patient and control the rotation speed of the impeller based on the activity level.

Example 75 is the blood circulation assist system of example 74, wherein the controller is configured to process the remote accelerometer output to measure a respiration rate and/or a diaphragm contraction and base the activity level on the respiration rate and/or the diaphragm contraction.

Example 76 is the blood circulation assist system of any one of example 46 through example 48, wherein the remote accelerometer output is indicative of accelerations in three orthogonal directions.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1-45. (canceled)

46. A blood circulation assist system, comprising:
a ventricular assist device (VAD) comprising an inlet, an outlet, an impeller, and a motor stator operable to rotate the impeller to pump a blood flow, wherein the inlet is configured for coupling with a ventricle of a patient to receive the blood flow from the ventricle, wherein the outlet is configured for coupling with a blood vessel of the patient to transfer the blood flow to the blood vessel; and
a controller configured to process a remote accelerometer output indicative of accelerations of the patient at a remote accelerometer location and control a rotation speed of the impeller based on the remote accelerometer output; and
a remote accelerometer configured to generate the remote accelerometer output, wherein the remote accelerometer is configured to be implanted in the patient at the remote accelerometer location, and wherein the remote accelerometer location is separated from the VAD to isolate the remote accelerometer from noise generated by the VAD.

47. The system of claim 46, wherein the remote accelerometer is configured for implantation in a pectoral region of the patient or abdominal wall region of the patient.

48. The system of claim 46, wherein:
the VAD further comprises power transistors that are used to control supply of electrical currents to windings of the motor stator; and
the remote accelerometer location is separated from the VAD to isolate the remote accelerometer from electrical switching noise generated by the power transistors.

49. The system of claim 46, wherein the controller is configured to:
process the remote accelerometer output to determine a heart rate of the patient; and
control the rotation speed of the impeller based on the heart rate.

50. The system of claim 46, wherein the controller is configured to process the remote accelerometer output to determine a valve opening timing of the patient.

51. The system of claim 46, wherein the controller is configured to process the remote accelerometer output to monitor for a valve disorder of the patient.

52. The system of claim 46, wherein the controller is configured to process the remote accelerometer output to determine a respiration rate of the patient.

53. The system of claim 46, wherein the controller is configured to process the remote accelerometer output to monitor for an occurrence of pump thrombosis in the VAD, an occurrence of an occlusion in the VAD, and/or an occurrence of an instability of the impeller.

54-55. (canceled)

56. The system of claim 46, wherein the controller is configured to process the remote accelerometer output to monitor an orientation of the patient.

57-61. (canceled)

62. The system of claim 46, wherein the controller is configured to process the remote accelerometer output to monitor for a fall of the patient.

63. (canceled)

64. The system of claim 46, wherein the controller is configured to process the remote accelerometer output to determine whether the patient is active or at rest.

65. The system of claim 46, wherein the controller is configured to process the remote accelerometer output to determine a wellness indicator for the patient.

66. The system of claim 46, wherein:
the controller is configured to process the remote accelerometer output to detect a cardiac cycle timing of the patient;
the cardiac cycle timing comprises a heart rate and a time of occurrence for each of one or more cardiac cycle events; and
the controller is configured to vary the rotation speed of the impeller in sync with the cardiac cycle timing.

67. (canceled)

68. The system of claim 66, wherein the controller is configured to:
process the remote accelerometer output to detect a time of occurrence of at least one heart sound; and
detect the cardiac cycle timing based on the time of occurrence of the at least one heart sound.

69. The system of claim 68 wherein the at least one heart sound comprises:
a sound of closure of at least one atrioventricular valve of the patient; and/or
a sound of closure of at least one semilunar valve of the patient.

70. The system of claim 66, wherein the controller is configured to vary the rotation speed of the impeller over a target cardiac cycle based on detected timing of one or more cardiac cycles that occur prior to the target cardiac cycle and/or the detected timing of the target cardiac cycle.

71. (canceled)

72. The system of claim 66, wherein the controller is configured to:
process the remote accelerometer output to measure an activity level of the patient; and
control the rotation speed of the impeller based on the activity level.

73. The system of claim 72, wherein the controller is configured to process the remote accelerometer output to measure a respiration rate for the patient and/or a diaphragm contraction for the patient, and base the activity level on the respiration rate and/or the diaphragm contraction.

74. The system of claim 46, wherein the controller is configured to:
process the remote accelerometer output to measure an activity level of the patient; and
control the rotation speed of the impeller based on the activity level.

75. The system of claim 74, wherein the controller is configured to:
process the remote accelerometer output to measure a respiration rate and/or a diaphragm contraction; and
base the activity level on the respiration rate and/or the diaphragm contraction.

76. (canceled)
